SEQUENCE LISTING (31 sequences)

SEQ ID NO: 1 - protein, 9 aa, Malus domestica
PTKQKHRSS

SEQ ID NO: 2 - protein, 305 aa, Malus domestica
MCPTKQKHRSSVAEHAGKCSRSEDSTTSTAHWISESVHGGSLRHVDLTTGTNGWASPPGDLFSLRAKNYLSKKQKGPAGDYLLQPCGTDWLRSSTKLENVLARPDNRVANALRKAQSQGRSLKSFIFAVNLQIPGKDQHSAVFYFATEDPIPAGSLLYRFVNGDDAFRNQRFKIVNRIVKGPWIVEKTVGNYSACLLGKALTCNYHRGPNYLEIDVDIASFGIAKAILRLALRYVTSVTIDMGFVVEAQAEDELPEKLVGAVRVCEMEMLSATVVEAPQTTVVARGLSFASKVNHHKSGDDDDDD

SEQ ID NO: 3 - protein, 18 aa, Malus domestica
MCPTKQKHRSSVAEHAGK

SEQ ID NO: 4 - protein, 299 aa, Artificial Sequence
  Synthesized
  misc_feature: Mdpcd1-297 peptide
MCPTKQKHRSSVAEHAGKCSRSEDSTTSTAHWISESVHGGSLRHVDLTTGTNGWASPPGDLFSLRAKNYLSKKQKGPAGDYLLQPCGTDWLRSSTKLENVLARPDNRVANALRKAQSQGRSLKSFIFAVNLQIPGKDQHSAVFYFATEDPIPAGSLLYRFVNGDDAFRNQRFKIVNRIVKGPWIVEKTVGNYSACLLGKALTCNYHRGPNYLEIDVDIASFGIAKAILRLALRYVTSVTIDMGFVVEAQAEDELPEKLVGAVRVCEMEMLSATVVEAPQTTVVARGLSFASKVNHHKSG

SEQ ID NO: 5 - protein, 296 aa, Populus trichocarpa
MHPTKQKHRSSGPESTTSRSSSTPGWITESINGGSLRHVDLHTGVNGWASPPGDLFSLRSKNYFIKKQKSPSGDYLLSPAGMDWLKSSTKLDNVLARPDNRVANALKKAQSQNKSLKSFIFAINLQVPGKDQHSAVFYFASEDPLPSDSLLYRFINGDDAFRNQRFKIVNRIEKGPWVVKKTVGNYSACLLGKALNINYHRGGNYFEIDVDVGSSKIAAAILHLALGYTAHVTIDMGFVVEAQTEEELPERLIGAIRVCQMEMSTARVVDSPSTGLARGSGFAKVEHHLSGDEEED

SEQ ID NO: 6 - DNA, 918 bp, Malus domestica
atgtgtccgacgaaacaaaagcaccggagctccgtcgcggaacacgccggaaaatgctccagatcggaggattctacaacctccaccgcccactggatctccgagtccgtccacggcggatccttgcgccacgtggacctcaccaccggaaccaacggctgggcgtcacctcccggggacctattctcgctccgcgccaagaactacttgtcgaaaaaacaaaaaggccccgccggcgactacctactgcagccgtgcggcactgactggctcagatccagcacgaaactcgagaacgtactcgctcgccccgataatcgcgtggccaacgcgcttcggaaggcacagtcgcaagggaggtccctgaagagcttcattttcgccgtgaatctccagattcccggcaaggaccagcacagcgccgttttctatttcgccacggaggatcctatccctgccggctcgcttctctaccggttcgtcaacggcgacgacgcgttccggaaccagcggttcaagatcgtgaaccggatcgtgaaagggccgtggatcgtcgagaagacggtagggaattacagtgcgtgcttgctggggaaggcgctgacgtgtaattaccacagaggacccaactacctcgagattgacgtcgatatcgcgagttttgggatcgcgaaagcgattctgcgacttgcattgaggtacgtgacgagcgtgacgatcgacatggggtttgtggtggaggcgcaggcggaggatgagctgcctgagaagttagtcggcgcggttcgggtatgcgagatggagatgttgtctgcaacggtcgtggaggcgccgcagacgacggtcgtagcgcggggattgagttttgcgtctaaggtgaatcatcacaagtccggcgacgacgacgacgacgattga

SEQ ID NO: 7 - DNA, 57 bp, Malus domestica
atgtgtccgacgaaacaaaagcaccggagctccgtcgcggaacacgccggaaaatag

SEQ ID NO: 8 - DNA, 900 bp, Artificial Sequence
  Synthesized
  misc_feature: Mdpcd1-297 encoding sequence
atgtgtccgacgaaacaaaagcaccggagctccgtcgcggaacacgccggaaaatgctccagatcggaggattctacaacctccaccgcccactggatctccgagtccgtccacggcggatccttgcgccacgtggacctcaccaccggaaccaacggctgggcgtcacctcccggggacctattctcgctccgcgccaagaactacttgtcgaaaaaacaaaaaggccccgccggcgactacctactgcagccgtgcggcactgactggctcagatccagcacgaaactcgagaacgtactcgctcgccccgataatcgcgtggccaacgcgcttcggaaggcacagtcgcaagggaggtccctgaagagcttcattttcgccgtgaatctccagattcccggcaaggaccagcacagcgccgttttctatttcgccacggaggatcctatccctgccggctcgcttctctaccggttcgtcaacggcgacgacgcgttccggaaccagcggttcaagatcgtgaaccggatcgtgaaagggccgtggatcgtcgagaagacggtagggaattacagtgcgtgcttgctggggaaggcgctgacgtgtaattaccacagaggacccaactacctcgagattgacgtcgatatcgcgagttttgggatcgcgaaagcgattctgcgacttgcattgaggtacgtgacgagcgtgacgatcgacatggggtttgtggtggaggcgcaggcggaggatgagctgcctgagaagttagtcggcgcggttcgggtatgcgagatggagatgttgtctgcaacggtcgtggaggcgccgcagacgacggtcgtagcgcggggattgagttttgcgtctaaggtgaatcatcacaagtccggctga

SEQ ID NO: 9 - DNA, 891 bp, Populus trichocarpa
atgcacccaaccaaacagaaacaccggagctccggtccggaatcaacaacctccagatcatcatcaacacccggttggatcaccgaatcgatcaacggtggatcacttcgccacgtggacttacacactggtgttaacggatgggcgtcaccgccaggtgatcttttctctctccgttcaaaaaactacttcatcaaaaaacagaaatccccctccggcgactacctactctcacccgccggtatggactggctcaaatctagcacaaaactcgacaacgtactcgctcgtccagataatcgcgtggcaaacgccttaaagaaagcacaatcccaaaataagtccctcaagtccttcatttttgctatcaaccttcaagtccccggtaaagaccaacacagcgccgttttctacttcgcatcggaagatcctctaccgtccgattccctcctatatagattcatcaacggcgatgatgcatttcggaatcaacggttcaagatcgtgaaccggattgaaaagggtccatgggtggtgaaaaaaacggtaggaaattacagcgcgtgtttattaggtaaagcgttaaatatcaattaccataggggtgggaattatttcgagattgatgttgatgttggcagctcgaaaattgctgcggcaattttgcatctcgcattggggtacacggcgcatgtcaccatcgatatggggtttgtcgtggaggcgcagacggaggaggagttgccggagaggttgattggggcaatcagggtttgtcagatggaaatgtcgacggcgcgtgttgttgattccccgtcgacgggtttggcgcgtgggtcggggtttgctaaggtggagcatcatttgtcaggcgatgaagaggaggattga

SEQ ID NO: 10 - DNA, 22 bp, Artificial Sequence
  Synthesized
  misc_feature: PCR Primer
atgtgtccaacaaagcaaaagc

SEQ ID NO: 11 - DNA, 22 bp, Artificial Sequence
  Synthesized
  misc_feature: PCR Primer
tcaatcgtcgtcgtcatcgtcg

SEQ ID NO: 12 - DNA, 22 bp, Artificial Sequence
  Synthesized
  misc_feature: PCR Primer
atgtgtccaacaaagcaaaagc

SEQ ID NO: 13 - DNA, 24 bp, Artificial Sequence
  Synthesized
  misc_feature: PCR Primer
tcagccggacttgtgatgattcac

SEQ ID NO: 14 - DNA, 20 bp, Artificial Sequence
  Synthesized
  misc_feature: PCR Primer
atgcacccaaccaaacagaa

SEQ ID NO: 15 - DNA, 20 bp, Artificial Sequence
  Synthesized
  misc_feature: PCR Primer
tcaatcctcctcttcatcgc

SEQ ID NO: 16 - DNA, 32 bp, Artificial Sequence
  Synthesized
  misc_feature: PCR Primer
cagcccatggatgtgtccaacaaagcaaaagc

SEQ ID NO: 17 - DNA, 32 bp, Artificial Sequence
  Synthesized
  misc_feature: PCR Primer
gaagtctagatcaatcgtcgtcgtcatcgtcg

SEQ ID NO: 18 - DNA, 32 bp, Artificial Sequence; identical to SEQ ID NO: 16
  Synthesized
  misc_feature: PCR Primer

SEQ ID NO: 19 - DNA, 34 bp, Artificial Sequence
  Synthesized
  misc_feature: PCR Primer
gaagtctagatcagccggacttgtgatgattcac

SEQ ID NO: 20 - DNA, 30 bp, Artificial Sequence
  Synthesized
  misc_feature: PCR Primer
cagcccatggatgcacccaaccaaacagaa

SEQ ID NO: 21 - DNA, 29 bp, Artificial Sequence
  Synthesized
  misc_feature: PCR Primer
gaagtctagatcaatcctcctcttcatcg

SEQ ID NO: 22 - DNA, 33 bp, Artificial Sequence
  Synthesized
  misc_feature: PCR Primer
agccaccatggatgtgtccaacaaagcaaaagc

SEQ ID NO: 23 - DNA, 33 bp, Artificial Sequence
  Synthesized
  misc_feature: PCR Primer
gtacctctagatcaatcgtcgtcgtcatcgtcg

SEQ ID NO: 24 - DNA, 33 bp, Artificial Sequence; identical to SEQ ID NO: 22
  Synthesized
  misc_feature: PCR Primer

SEQ ID NO: 25 - DNA, 31 bp, Artificial Sequence
  Synthesized
  misc_feature: PCR Primer
gtacctctagatcagccggacttgtgatgat

SEQ ID NO: 26 - DNA, 31 bp, Artificial Sequence
  Synthesized
  misc_feature: PCR Primer
agccaccatgcacccaaccaaacagaaacac

SEQ ID NO: 27 - DNA, 31 bp, Artificial Sequence
  Synthesized
  misc_feature: PCR Primer
gtacctctagatcaatcctcctcttcatcgc

SEQ ID NO: 28 - protein, 302 aa, Arabidopsis thaliana
MSPSKQRHRSSTGENKSKPVRSGSSSAISEWITESTNGGSLRRVDPDTGTDGWASPPGDVFSLRSDSYLSKKQKTPAGDYLLSPAGMDWLKSSTKLENVLARPDNRVAHALRKAQSRGQSLKSFIFAVNLQIPGKDHHSAVFYFATEEPIPSGSLLHRFINGDDAFRNQRFKIVNRIVKGPWVVKAAVGNYSACLLGKALTCNYHRGPNYFEIDVDISSSAIATAILRLALGYVTSVTIDMGFLAEAQTEEELPERLIGAVRVCQMEMSSAFVVDAPPPQQLPSQPCRTLSSAKVNHDEDED

SEQ ID NO: 29 - protein, 268 aa, Artificial Sequence
  Synthesized
  misc_feature: Mdpcd1-268 peptide
WISESVHGGSLRHVDLTTGTNGWASPPGDLFSLRAKNYLSKKQKGPAGDYLLQPCGTDWLRSSTKLENVLARPDNRVANALRKAQSQGRSLKSFIFAVNLQIPGKDQHSAVFYFATEDPIPAGSLLYRFVNGDDAFRNQRFKIVNRIVKGPWIVEKTVGNYSACLLGKALTCNYHRGPNYLEIDVDIASFGIAKAILRLALRYVTSVTIDMGFVVEAQAEDELPEKLVGAVRVCEMEMLSATVVEAPQTTVVARGLSFASKVNHHKSG

SEQ ID NO: 30 - protein, 307 aa, Artificial Sequence
  Synthesized
  misc_feature: Mdpcd1-307 peptide
MCPTKQKHRSSVAEHAGKCSRSEDSTTSTAHWISESVHGGSLRHVDLTTGTNGWASPPGDLFSLRAKNYLSKKQKGPAGDYLLQPCGTDWLRSSTKLENVLARPDNRVANALRKAQSQGRSLKSFIFAVNLQIPGKDQHSAVFYFATEDPIPAGSLLYRFVNGDDAFRNQRFKIVNRIVKGPWIVEKTVGNYSACLLGKALTCNYHRGPNYLEIDVDIASFGIAKAILRLALRYVTSVTIDMGFVVEAQAEDELPEKLVGAVRVCEMEMLSATVVEAPQTTVVARGLSFASKVNHHKSGDDDDDDDD

SEQ ID NO: 31 - DNA, 924 bp, Artificial Sequence
  Synthesized
  misc_feature: Mdpcd1-307 encoding sequence
atgtgtccgacgaaacaaaagcaccggagctccgtcgcggaacacgccggaaaatgctccagatcggaggattctacaacctccaccgcccactggatctccgagtccgtccacggcggatccttgcgccacgtggacctcaccaccggaaccaacggctgggcgtcacctcccggggacctattctcgctccgcgccaagaactacttgtcgaaaaaacaaaaaggccccgccggcgactacctactgcagccgtgcggcactgactggctcagatccagcacgaaactcgagaacgtactcgctcgccccgataatcgcgtggccaacgcgcttcggaaggcacagtcgcaagggaggtccctgaagagcttcattttcgccgtgaatctccagattcccggcaaggaccagcacagcgccgttttctatttcgccacggaggatcctatccctgccggctcgcttctctaccggttcgtcaacggcgacgacgcgttccggaaccagcggttcaagatcgtgaaccggatcgtgaaagggccgtggatcgtcgagaagacggtagggaattacagtgcgtgcttgctggggaaggcgctgacgtgtaattaccacagaggacccaactacctcgagattgacgtcgatatcgcgagttttgggatcgcgaaagcgattctgcgacttgcattgaggtacgtgacgagcgtgacgatcgacatggggtttgtggtggaggcgcaggcggaggatgagctgcctgagaagttagtcggcgcggttcgggtatgcgagatggagatgttgtctgcaacggtcgtggaggcgccgcagacgacggtcgtagcgcggggattgagttttgcgtctaaggtgaatcatcacaagtccggcgacgacgacgacgacgacgacgattga

(19) United States
(12) Patent Application Publication
(10) Pub. No.: US 2023/0391836 A1
(43) Pub. Date: Dec. 7, 2023

(54) AMINO ACID SEQUENCE THAT CAN DESTROY CELLS, AND RELATED NUCLEOTIDE SEQUENCE AND RELATED USES THEREOF

(71) Applicant: Yuezhi WANG, Hangzhou, Zhejiang (CN)

(72) Inventor: Yuezhi WANG, Hangzhou, Zhejiang (CN)

(21) Appl. No.: 18/245,095

(22) PCT Filed: Sep. 13, 2021
(86) PCT No.: PCT/CN2021/118063

§ 371 Date: Mar. 13, 2023

(30) Foreign Application Priority Data
Sep. 11, 2020 (CN) 202010956519.2

(51) Int. Cl. C07K 14/415; C12N 15/63; A61P 35/00

(52) U.S. Cl. CPC C07K 14/415; C12N 15/63; A61P 35/00

(57) ABSTRACT

An amino acid sequence that can destroy cells, a nucleotide sequence encoding and expressing the corresponding amino acid sequence, and the related uses of the amino acid sequence and the nucleotide sequence are provided. The amino acid sequence has the amino acid sequence shown in SEQ ID NO: 1 and/or SEQ ID NO: 29.

### INCORPORATION OF SEQUENCE LISTING

This application contains a sequence listing submitted in Computer Readable Form (CRF). The CFR file containing the sequence listing entitled “PBA408-0110_ST25.txt”, which was created on Mar. 13, 2023, and is 26,249 bytes in size. The information in the sequence listing is incorporated herein by reference in its entirety.

### TECHNICAL FIELD

The present invention belongs to the field of biotechnology, and in particular to an amino acid sequence that can destroy cells, a nucleotide sequence encoding the corresponding amino acid sequence, and related uses of the amino acid sequence and the nucleotide sequence.

### BACKGROUND

The functions realized by the expression of some proteins can interact with the key proteins related to the cell functions to destroy the cells, which can be used for tumor treatment.

For example, ARF1 (ADP-ribosylation factor 1) has been identified as the key molecular switch for vesicle formation in the Golgi apparatus secretory transport pathway (Kahn et al., The Journal of biological chemistry. 1992, 267:13039-13046; Beck et al., The Journal of Cell Biology. 2010, 194:765-777). In all eukaryotes, ARF1 is conservative in its function and sequence characteristics (Cevher-Keskin, Int. J. Mol. Sci. 2013, 14, 18181-18199). A number of studies have reported that the expression of ARF1 is related to the replication and proliferation of tumor cells and has been proved to be a molecular switch for the replication and proliferation of cancer cells (Boulay et al., The Journal of biological chemistry. 2008, 283:36425-36434; Hashimoto et al., Proceedings of the National Academy of Sciences of the United States of America. 2004, 101:6647-6652; Schlienger et al., Oncotarget, 2016, 7: 11811-11827; Davis et al., Oncotarget, 2016, 7:39834-39845). ARF1 has been studied as a key molecular target for the treatment and diagnosis of related cancers (Schlienger et al., Oncotarget, 2016, 7: 11811-11827; Davis et al., Oncotarget, 2016, 7:39834-39845; Ohashi et al., The Journal of Biological Chemistry, 2012, 287, 3885-3897). Compound AMF-26 ((2E,4E)-5-((1 S,2 S,4aR,6R,7 S,8S,8a5)-7-hydroxy-2,6,8-trimethyl-1,2,4a, 5,6,7,8,8a-octahydronaphthalen-1-yl)-2-methyl-N-(pyridin-3-yl-methyl)penta-2,4-dienamide) induces the disintegration of Golgi apparatus, the cell necrosis and the inhibition of cell growth by targeting ARF1, and oral administration of AMF-26 has good therapeutic effect on human breast cancer (Ohashi et al., 2012, J Biol Chem 287(6): 3885-3897). Eukaryotic translation elongation factor eEF1α is highly expressed and plays a key role in tumors (including breast cancer, ovarian cancer and lung cancer, etc.) and many human diseases (Abbas et al., Front. Oncol., 2015, 5:75). Targeted inhibition of eEf1a by narciclasine can cause cancer cell apoptosis, which can effectively treat melanoma (Van Goietsenoven et al., FASEB J. 2010, 24(11):4575-84). Compared with chemical drugs, gene therapy has the potential of high efficiency in the preparation of drugs and treatment. Therefore, the destruction of cells can be started through the interaction with ARF1 or eEF1a, thereby achieving the effect of cancer treatment.

### SUMMARY OF THE INVENTION

The present invention provides an amino acid sequence that can destroy cells, a nucleotide sequence encoding the corresponding amino acid sequence, and related uses of the amino acid sequence and the nucleotide sequence to achieve the destruction of cells and provide a new solution for tumor treatment. For these purposes, the present invention provides following technical solutions.

In the first aspect of the present invention, it provides an amino acid sequence that can destroy cells, wherein the destruction of the cells refers to the effect of triggering the collapse of the cell membrane system to destroy the cells, and can further comprise the effect of tissue damage caused by cell destruction.

The amino acid sequence provided in the present invention also has following characteristic: wherein the amino acid sequence comprises the amino acid sequence shown in SEQ ID NO: 1 and/or SEQ ID NO: 29.

The amino acid sequence provided in the present invention also has following characteristic: wherein the amino acid sequence is an Mdpcd1-303 protein fragment with the amino acid sequence shown in SEQ ID NO: 2, or a derived protein or homologous protein of the Mdpcd1-303 protein fragment derived from SEQ ID NO: 2 with the same activity as the amino acid residue sequence of SEQ ID NO: 2, which is formed by substituting, deleting or adding one or more amino acid residues in the amino acid residue sequence of SEQ ID NO: 2.

The amino acid sequence provided in the present invention also has following characteristic: wherein the derived protein comprises an Mdpcd1-18 protein fragment, an Mdpcd1-297 protein fragment and an Mdpcd1-307 protein fragment, the homologous protein comprises a Ptpcd1-296 protein fragment, and the Mdpcd1-18 protein fragment has the amino acid sequence shown in SEQ ID NO: 3; the Mdpcd1-297 protein fragment has the amino acid sequence shown in SEQ ID NO: 4; the Mdpcd1-307 protein fragment has the amino acid sequence shown in SEQ ID NO: 30, and the Ptpcd1-296 protein fragment has the amino acid sequence shown in SEQ ID NO: 5.

In the second aspect of the present invention, it provides a nucleotide sequence for encoding the amino acid sequence that can destroy cells, wherein the amino acid sequence is the above amino acid sequence.

The nucleotide sequence provided in the present invention also has following characteristic: wherein the nucleotide sequence is used for encoding any one of the Mdpcd1-303 protein fragment, a derived protein of the protein fragment, and a homologous protein of the protein fragment.

The nucleotide sequence provided in the present invention also has following characteristic: wherein the nucleotide sequence is used for encoding the Mdpcd1-303 protein fragment, and the nucleotide sequence is the nucleotide sequence shown in SEQ ID NO: 6.

The nucleotide sequence provided in the present invention also has following characteristic: wherein the derived protein of the Mdpcd1-303 protein fragment comprises an Mdpcd1-18 protein fragment, an Mdpcd1-297 protein fragment and an Mdpcd1-307 protein fragment, the homologous protein comprises a Ptpcd1-296 protein fragment, and wherein the nucleotide sequence for encoding the Mdpcd1-18 protein fragment is the nucleotide sequence shown in SEQ ID NO: 7; the nucleotide sequence for encoding the Mdpcd1-297 protein fragment is the nucleotide sequence shown in SEQ ID NO: 8; the nucleotide sequence for encoding the Mdpcd1-307 protein fragment is the nucleotide sequence shown in SEQ ID NO: 31, and the nucleotide sequence for encoding the Ptpcd1-296 protein fragment is the nucleotide sequence shown in SEQ ID NO: 9.

In the third aspect of the present invention, it provides a vector (preferably an expression vector), which comprises the nucleotide sequence described above.

In the fourth aspect of the present invention, it provides a use of the amino acid sequence, or the nucleotide sequence, or the vector in destroying cells, wherein the amino acid sequence is the above amino acid sequence; the nucleotide sequence is the above nucleotide sequence; and the vector is the above vector.

The present invention further provides a use of an amino acid sequence, or a nucleotide sequence, or a vector in tumor treatment, wherein the amino acid sequence is the above amino acid sequence; the nucleotide sequence is the above nucleotide sequence; and the vector is the above vector.

The present invention further provides a use of an amino acid sequence, or a nucleotide sequence, or a vector in the manufacture of a composition for destroying cells, wherein the amino acid sequence is the above amino acid sequence; the nucleotide sequence is the above nucleotide sequence; and the vector is the above vector.

The present invention further provides a use of an amino acid sequence, or a nucleotide sequence, or a vector in the manufacture of a medicament for use in tumor treatment, wherein the amino acid sequence is the above amino acid sequence; the nucleotide sequence is the above nucleotide sequence; and the vector is the above vector.

In the fifth aspect of the present invention, it provides a composition comprising an amino acid sequence, or a nucleotide sequence, or a vector, wherein the amino acid sequence is the above amino acid sequence; the nucleotide sequence is the above nucleotide sequence; and the vector is the above vector.

In another preferred embodiment, the composition further comprises a pharmaceutically acceptable carrier.

The present invention further provides a pharmaceutical composition comprising an amino acid sequence, or a nucleotide sequence, or a vector, wherein the amino acid sequence is the above amino acid sequence; the nucleotide sequence is the above nucleotide sequence; and the vector is the above vector.

In the sixth aspect of the present invention, it provides a method for destroying cells, comprising the following step: (a) contacting the cells to be destroyed with the destructive polypeptide of the first aspect of the present invention, so as to collapse the cell membrane system of the cells, thereby destroying the cells.

In another preferred embodiment, the cells are mammalian cells.

In another preferred embodiment, the cells are tumor cells.

In another preferred embodiment, in step (a), a nucleic acid or a vector expressing the destructive polypeptide is introduced into the cells, thereby expressing or overexpressing the destructive polypeptide in the cells.

In another preferred embodiment, the method further comprises the following step: (b) detecting the integrity of the cell membrane and/or the survival of the cells in step (a), thereby qualitatively or quantitatively determining the destruction of the cells.

In another preferred embodiment, the method is a non-therapeutic and non-diagnostic method.

In another preferred embodiment, the method is an in vitro method.

In another preferred embodiment, the method is a therapeutic method.

Preferably, Mdpcd1-303 and its derived protein or homologous protein comprising the amino acid sequence shown in SEQ ID NO: 1 and/or SEQ ID NO: 29 are obtained in the present invention. These proteins have been proved to trigger the collapse of the cell membrane system and achieve the effect of destroying cells. It has also been proved that the nucleotide sequence encoding the amino acid sequence can have therapeutic effect on tumors, and the corresponding amino acid can also have therapeutic effect on tumors.

Using any one of expression vectors that can guide the proper expression (including overexpression) of exogenous genes in tumor cells, the encoding nucleotide sequence of the Mdpcd1-303 protein fragment or its derived protein fragment Mdpcd1-18 or its derived protein fragment Mdpcd1-297 or its derived protein fragment Mdpcd1-307 or its homologous protein fragment Ptpcd1-296 provided in the present invention can be introduced into tumor cells, which can change the life process of the tumor cells, initiate the programmed death of the tumor cells, realize the inhibition and killing of tumor tissue, and enhance the immune function of the organism. When using the vector, any kind of enhanced promoter or inducible promoter or tumor cell-specific promoter can be added before the transcription initiation nucleotide of the vector. The expression vector comprising the above nucleotide sequence of the present invention can be used to transfect tumor cells by using various virus vectors such as adenovirus, retrovirus, adeno-associated virus, vaccinia virus, herpes virus, lentivirus and so on as a gene introduction system, and can also be used to transfect tumor cells by using naked plasmid DNA, liposome, cationic polymer and other methods.

It should be understood that within the scope of the present invention, the above-mentioned technical features of the present invention and the technical features specifically described in the following (such as the embodiments) can be combined with each other to form a new or preferred technical solution, which are not redundantly repeated one by one due to space limitation.

### DESCRIPTION OF THE DRAWINGS

FIG. 1 shows the results of the immersion test with the transient expression vector involved in Example 9, which comprises the nucleotide sequence of the Mdpcd1-303 protein fragment.

FIG. 2 shows the results of the immersion test with the transient expression vector involved in Example 9, which comprises the nucleotide sequence of the Mdpcd1-18 protein fragment.

FIG. 3 shows the results of the immersion test with the transient expression vector involved in Example 9, which comprises the nucleotide sequence of the Mdpcd1-297 protein fragment.

FIG. 4 shows the results of the immersion test with the transient expression vector involved in Example 9, which comprises the nucleotide sequence of the Ptpcd1-296 protein fragment.

FIG. 5 shows the changes of tobacco leaf cells after the immersion test with the transient expression vector involved in Example 9, which comprises the nucleotide sequence of the Mdpcd1-303 protein fragment.

FIG. 6 shows the results of the immersion test with the transient expression vector involved in Example 9, which comprises the nucleotide sequence of the Atpcd1 protein.

FIG. 7 shows the identification of protein interaction between the Mdpcd1-303 protein involved in Example 10 and the ARF1 by yeast two-hybrid (QDO medium).

FIG. 8 shows the subcutaneous tumor growth curves of mice treated with Adc68-Mdpcd1-303 injection and tumor-bearing mice as control group in Example 11. **P<0.01.

FIG. 9 shows the subcutaneous tumor growth curves of mice treated with Adc68-Mdpcd1-18 injection and tumor-bearing mice as control group in Example 12. **P<0.01.

FIG. 10 shows the subcutaneous tumor growth curves of mice treated with Adc68-Mdpcd1-297 injection and tumor-bearing mice as control group in Example 13. **P<0.01.

FIG. 11 shows the subcutaneous tumor growth curves of mice treated with Adc68-Ptpcd1-296 injection and tumor-bearing mice as control group in Example 14. **P<0.01.

FIG. 12 shows the results of the immersion test with the transient expression vector involved in Example 17, which comprises the nucleotide sequence of the Mdpcd1-307 protein fragment.

FIG. 13 shows the cell apoptosis detection results of the inhibition effect of the Mdpcd1-307 protein involved in Example 18 on the activity of SMMC-7721 (human hepatoma cells, the same below) by flow cytometry.

FIG. 14 shows the detection results of the inhibition effect of the Mdpcd1-307 protein involved in Example 18 on the infectivity of SMMC-7721.

FIG. 15 shows the TUNEL staining results showing that the Mdpcd1-307 protein involved in Example 19 promotes the apoptosis rate of SMMC-7721 tumor cells.

FIG. 16 shows the number of differentially expressed genes in SMMC-7721 tumor regulated by the Mdpcd1-307 protein involved in Example 19.

FIG. 17 shows the functional enrichment clustering of metabolic pathway of differentially expressed genes in SMMC-7721 tumor regulated by the Mdpcd1-307 protein involved in Example 19.

FIG. 18 shows the inhibition of the Mdpcd1-307 protein involved in Example 19 on SMMC-7721 tumor in mice.

FIG. 19 shows the interference on ARF1 expression inhibits the cell necrosis caused by the overexpression of the Mdpcd1-303.

FIG. 20 shows the identification of protein interaction between the Mdpcd1-303 protein involved in Example 21 and the eEF1a by yeast-two hybrid.

FIG. 21 shows the interference on eEF1a expression inhibits the cell necrosis caused by the overexpression of the Mdpcd1-303.

FIG. 22 shows the overexpression of eEF1a and Mdpcd1-307 initiates the cell necrosis.

### MODES FOR CARRYING OUT THE INVENTION

After extensive and intensive research, the inventors have unexpectedly developed a destructive polypeptide with unique functions derived from plants for the first time. When the destructive polypeptide is overexpressed, it can trigger the collapse of the cell membrane system and achieve the effect of destroying cells. Specifically, the destructive polypeptide Mdpcd1-303 of the present invention is derived from plants and is a plant-specific gene, and there is no homologous gene in animals; when the protein encoded by this gene is overexpressed (such as through the 35S promoter initiating gene expression), it can cause cell death of tobacco leaves.

In addition, the research of the present invention also shows that the interaction protein of the Mdpcd1-303 encoding protein (such as ARF1 or eEF1a) plays a key role in the cell death of tobacco leaves initiated by the gene; ARF1 and eEF1a are both conservative in animals and plants, and are closely related to multiple tumorigenesis.

Mdpcd1-307 is an allele of Mdpcd1-303. The protein encoded by the Mdpcd1-307 cannot cause cell death in tobacco leaves when overexpressed (through the 35S promoter initiating gene expression), but when the Mdpcd1-307 is co-expressed with ARF1, it can cause cell death in tobacco leaves; similarly, when the Mdpcd1-307 is co-expressed with eEF1a, it can also cause cell death in tobacco leaves.

The results of hepatoma cell experiment in vitro show that the Mdpcd1-303 can significantly increase the apoptosis rate of hepatoma cells; and in vivo experiments in mice show that the Mdpcd1-303 can significantly inhibit the growth of tumor tissue. The allele or core sequence of the Mdpcd1-303 also has similar functions.

Terms

In order to make it easier to understand the present disclosure, certain terms are firstly defined. As used in this application, unless expressly stated otherwise herein, each of the following terms shall have the meaning given below.

The term “about” may refer to a value or composition within an acceptable error range of a particular value or composition determined by a person of ordinary skill in the art, which will depend in part on how the value or composition is measured or determined.

The term “administration” refers to a physical introduction of the product of the present invention into the subject by using any of the various methods and delivery systems known to those skilled in the art, including intravenous, intratumor, intramuscular, subcutaneous, intraperitoneal, spinal or other parenteral administration routes, such as through injection or infusion.

Destructive Polypeptide Mdpcd1-303 and its Derived Protein or Homologous Protein

As used herein, the terms “destructive polypeptide”, “polypeptide of the present invention” and “destructive polypeptide of the present invention” can be used interchangeably, referring to the Mdpcd1-303 and its derived protein or homologous protein. It should be understood that the terms comprise wild type or mutant type. In addition, the terms also comprise a full-length protein or its functional fragment.

A preferred destructive protein is the Mdpcd1-303 and its derived protein (such as Mdpcd1-18, Mdpcd1-297, and Mdpcd1-307) or homologous protein (such as Ptpcd-296) comprising the amino acid sequence shown in SEQ ID NO: 1 and/or SEQ ID NO: 29.

The experiments of the present invention show that when the protein is overexpressed or in large quantities, it can trigger the collapse of the cell membrane system and achieve the effect of destroying cells. It has also been proved that the nucleotide encoding the amino acid sequence of the protein can have therapeutic effects on tumors, and the corresponding amino acid can also have therapeutic effects on tumors.

In addition, by using any one of vectors that can guide the proper expression (including overexpression) of exogenous genes in tumor cells, the encoding nucleotide sequence of the Mdpcd1-303 protein fragment or its derived protein fragment Mdpcd1-18 or its derived protein fragment Mdpcd1-297 or its derived protein fragment Mdpcd1-307 or its homologous protein fragment Ptpcd1-296 provided in the present invention can be introduced into tumor cells, which can change the life process of the tumor cells, initiate the programmed death of the tumor cells, realize the inhibition and killing of tumor tissue, and enhance the immune function of the organism.

Destroying Cells

As used herein, the term “destroy cells” or “destroying cells” can be used interchangeably, referring to the effect of triggering the collapse of the cell membrane system so as to destroy cells, and can further comprise the effect of tissue damage caused by cell destruction.

In another preferred embodiment, the destruction of the cells refers to the destruction or damage to plant cells, animal cells, cancer cells, etc., or combinations thereof, or corresponding tissue structure thereof.

In another preferred embodiment, the cells (including cells to be destroyed, being destroyed or having been destroyed) include (but are not limited to): plant cells and animal cells. Preferably, the cells are human and non-human mammalian cells.

In another preferred embodiment, the cells are pathological cells, such as tumor cells.

In another preferred embodiment, the tumor cells include (but are not limited to): ovarian cancer cells, lung cancer cells, pancreatic cancer cells, liver cancer cells, gastric cancer cells, breast cancer cells, nasopharyngeal cancer cells, esophageal cancer cells, colorectal cancer cells, cervical cancer cells, leukemia and lymphoma cells, etc.

In another preferred embodiment, the destruction of the cells refers to the destruction and damage to tobacco leaf cells and tobacco leaves.

In another preferred embodiment, the destruction of the cells refers to the destruction and damage to liver cancer cells and hepatocellular carcinoma xenografts.

Vector

The present invention further provides a vector comprising the amino acid sequence or nucleotide sequence of the present invention. Vectors derived from retroviruses, such as adenoviruses, are suitable tools for long-term gene transfer, because they allow transgenes to be stably integrated into the cell genome for a long time and replicate with the replication of the genome of daughter cells. The efficiency of adenovirus vectors is high, and the efficiency of in vitro experiments is usually close to 100%; adenovirus vectors can transduce different types of human tissue cells, regardless of whether the target cells are divided cells; it is easy to prepare virus vectors with high titer; and adenovirus vectors will not be integrated into the host cell genome after entering the cells, but will only be expressed transiently, with high safety. Therefore, adenovirus vectors have more and more applications in clinical trials of gene therapy, and become the most widely used and promising virus vectors after retroviral vectors.

Generally, the amino acid sequence or nucleotide sequence of the present invention can be connected to the downstream of a promoter and incorporated into an expression vector by conventional operation. The vector can be integrated into the eukaryotic genome and then replicated. Typical cloning vectors include transcription and translation terminators, initial sequences and promoters that can be used to regulate the expression of desired nucleic acid sequences.

The expression vector of the present invention can also be used for standard gene delivery schemes, nucleic acid immunization and gene therapy. The methods of gene delivery are known in the art. See, for example, U.S. Pat. Nos. 5,399,346, 5,580,859 and 5,589,466, which are incorporated herein by reference in their entirety.

The amino acid sequence or nucleotide sequence can be cloned into many types of vectors. For example, the amino acid sequence or nucleotide sequence can be cloned into vectors including but not limited to plasmids, bacteriophages, bacteriophage derivatives, animal viruses and cosmid vectors. Specific vectors of interest include expression vectors, replication vectors, etc.

Furthermore, the expression vectors can be provided to cells in the form of virus vectors. Virus vector technology is well known in this field and described in such as Molecular Cloning: A Laboratory Manual (Sambrook et al., Cold Spring Harbor Laboratory, New York, 2001) and other virology and molecular biology manuals. Viruses that can be used as vectors include but are not limited to retroviruses, adenoviruses, adeno-associated viruses, herpes viruses and lentiviruses. Generally, a suitable vector comprises at least one origin of replication, promoter sequence, convenient restriction enzyme site and one or more selectable markers (for example, WO01/96584; WO01/29058; and U.S. Pat. No. 6,326,193) that play a role in the organism.

Many virus-based systems have been developed and used for gene transduction in mammalian cells. For example, retroviruses provide a convenient platform for gene delivery systems. The selected genes can be inserted into vectors and packaged into retroviral particles using the technology known in the art. The recombinant viruses can then be isolated and transmitted to the target cells in vivo or in vitro. Many retroviral systems are known in the art. In some embodiments, adenovirus vectors are used. Many DNA virus systems are known in the art. Many adenovirus vectors are known in the art.

Additional promoter elements, such as enhancers, can regulate the frequency of transcription initiation. Generally, these elements are located in the 30-110 bp region upstream of the initiation site, although recently it has been shown that many promoters also comprise functional elements downstream of the initiation site. The spacing between the promoter elements is often flexible to maintain the promoter function when the element is inverted or moved relative to another element. In the thymidine kinase (tk) promoters, the spacing between the promoter elements can be increased by bp before the activity begins to decline. Depending on the promoter, it shows that a single element can work cooperatively or independently to initiate transcription.

An example of suitable promoters is the cytomegalovirus (CMV) promoter sequence. The promoter sequence is a strong constitutive promoter sequence that can drive the high-level expression of any polynucleotide sequence operably connected to it. Another example of suitable promoters is the elongation growth factor-1a (EF-1a). However, other constitutive promoter sequences can also be used, including but not limited to simian virus 40 (SV40) early promoter, mouse mammary tumor virus (MMTV), human immunodeficiency virus (HIV) long terminal repeat (LTR) promoter, MoMuLV promoter, avian leukemia virus promoter, Epstein-Barr virus (EBV) immediate-early promoter, Rous sarcoma virus promoter, and human gene promoter, such as but not limited to actin promoter, myosin promoter, heme promoter and creatine kinase promoter. Further, the present invention shall not be limited to the application of constitutive promoters. Inducible promoters are also considered as a part of the present invention. The use of inducible promoters provides a molecular switch that can initiate the expression of polynucleotide sequences connected to inducible promoters when needed, or turn off the expression when not needed. Examples of inducible promoters include but are not limited to metallothionein promoters, glucocorticoid promoters, progesterone promoters and tetracycline promoters.

The expression vectors introduced into the cells can also comprise any one or both of the selectable marker genes or reporter genes, so as to identify and select the expression cells from the transfected or infected population of cells through the virus vectors. In other aspects, the selectable markers can be carried on a single fragment of DNA and used for co-transfection procedures. The flanks of selectable marker genes and reporter genes can have appropriate regulatory sequences so that they can be expressed in host cells. Useful selectable marker genes include, for example, antibiotic resistance genes, such as neomycin, etc.

Methods for introducing genes into cells and expressing genes in cells are known in the art. In the content of expression vectors, vectors can be easily introduced into host cells, such as mammalian cells (such as human T cells), bacteria cells, yeast cells or insect cells by any method in the art. For example, expression vectors can be transferred into host cells by physical, chemical or biological means.

The physical methods for introducing polynucleotides into host cells include calcium phosphate precipitation, cationic complex transfection, lipid transfection, particle bombardment, microinjection, electroporation, etc. Methods for producing cells comprising vectors and/or exogenous nucleic acids are well known in the art. See, for example, Molecular Cloning: A Laboratory Manual (Sambrook et al., Cold Spring Harbor Laboratory, New York, 2001). The optimal methods for introducing polynucleotides into host cells are liposome transfection and cationic complex polyethyleneimine transfection.

Biological methods for introducing polynucleotides into host cells include the use of DNA and RNA vectors. Virus vectors, especially retroviral vectors, have become the most widely used method for inserting genes into mammalian cells, such as human cells. Other virus vectors can be derived from lentivirus, poxvirus, herpes simplex virus I, adenovirus and adeno-associated virus, etc. See, for example, U.S. Pat. Nos. 5,350,674 and 5,585,362.

The chemical means of introducing polynucleotides into host cells include colloidal dispersion systems, such as macromolecular complexes, nanocapsules, microspheres and beads; and lipid-based systems, including oil-in-water emulsions, micelles, mixed micelles and liposomes. An exemplary colloidal system used as a delivery vehicle in vitro and in vivo is a liposome (e.g., an artificial capsule).

In the case of non-viral delivery system, the exemplary delivery tool is a liposome. Lipid preparations are considered to be used to introduce nucleic acids into host cells (in vitro, ex vivo or in vivo). On the other hand, the nucleic acids can be associated with lipids. The nucleic acids associated with lipids can be encapsulated in the aqueous interior of the liposome, dispersed in the lipid bilayer of the liposome, attached to the liposome by the connecting molecules associated with both the liposome and the oligonucleotides, trapped in the liposome, compounded with the liposome, dispersed in the solution containing lipids, mixed with lipids, combined with lipids, contained in lipids as a suspension, contained in micelles or compounded with micelles, or associated with lipids in other ways. The lipid, lipid/DNA or lipid/expression vector associated with the composition is not limited to any specific structure in the solution. They can also be simply dispersed in the solution and may form aggregates with uneven size or shape. Lipids are lipid substances, which can be naturally occurring or synthesized. For example, lipids include fat droplets, which naturally occur in the cytoplasm and such compounds containing long-chain aliphatic hydrocarbons and their derivatives such as fatty acids, alcohols, amines, aminoalcohols and aldehydes.

In the embodiment of the present invention, the vector is an adenovirus vector.

Pharmaceutical Composition and Mode of Administration

The present invention also provides a pharmaceutical composition comprising any molecular entity that promotes the expression or activity of the amino acid sequence and/or nucleotide sequence that can destroy cells, or the amino acid sequence and/or nucleotide sequence that can destroy cells, or an expression vector of a molecular entity promoting the expression and activity of the amino acid sequence and/or nucleotide sequence that can destroy cells, as well as other pharmaceutically acceptable carriers.

The pharmaceutical composition of the present invention usually contains 10⁸-10⁹ PFU adenovirus particles.

As used herein, the term “pharmaceutically acceptable carrier” refers to the carrier used for the administration of therapeutic agents, including various excipients and diluents. They themselves are not active ingredients which are necessary, and there is no excessive toxicity after administration. Suitable carriers are well known to those skilled in the art. The pharmaceutically acceptable carrier in the composition can contain liquid, such as water, saline and buffer. In addition, there may also be auxiliary substances in these carriers, such as fillers, lubricants, glidants, wetting agents or emulsifiers, pH buffering substances, etc. The carrier can also contain cell transfection reagents.

Generally, the pharmaceutical composition of the present invention can be obtained by mixing the expression vector with a pharmaceutically acceptable carrier.

The mode of administration of the composition of the present invention is not particularly limited. Representative examples include but are not limited to: intravenous injection, subcutaneous injection, brain injection, intrathecal injection, and spinal injection, etc.

Therapeutic Application

The molecular entity promoting the expression or activity of the amino acid sequence and/or nucleotide sequence that can destroy cells, or the amino acid sequence and/or nucleotide sequence that can destroy cells, or the expression vector promoting the expression and activity of the amino acid sequence and/or nucleotide sequence that can destroy cells of the present invention, can be used to prepare a medicament that destroy tumor cells, inhibit replication and proliferation of tumor cells, change the life process of tumor cells, initiate the programmed death of tumor cells, realize the inhibition and killing of tumor tissue, and enhance the immune function of the organism. Moreover, because the cell-destructive amino acid sequence and/or the protein encoded by the nucleotide sequence of the present invention interacts with the GTP enzyme (GTPase) family (such as ARF1 or eEF1a), and the gene mutations in the enzyme family are related to multiple carcinogenesis, the present invention can also be used to prepare potential drugs for the treatment of cancers related to GTPase gene mutations.

### Advantages of the Invention

1. The present invention obtains the amino acid sequence comprising SEQ ID NO: 1 and/or SEQ ID NO: 29, the nucleotide sequence encoding the amino acid sequence, and Mdpcd1-303 and its derived protein or homologous protein comprising the amino acid sequence shown in SEQ ID NO: 1 and/or SEQ ID NO: 29. Mdpcd1-303 gene is derived from plants and is a plant-specific gene, and there is no homologous gene in animals. When the protein encoded by this gene is overexpressed (through the 35S promoter initiating gene expression), it can cause cell death, and the interaction protein (such as ARF1 or eEF1a) of the protein encoded by Mdpcd1-303 plays a key role in the cell death initiated by this gene. Because ARF1 and eEF1a are conservative in animals and plants, and are closely related to many kinds of tumors, the amino acid sequence comprising SEQ ID NO: 1 and/or SEQ ID NO: 29, the nucleotide sequence encoding the amino acid sequence, and the vector comprising the nucleotide sequence of the present invention can cause cell death by interacting with ARF1 and eEF1a in animals and plants, extending the scope of action of plant-specific genes to animals.

2. Mdpcd1-307 is an allele of Mdpcd1-303. The protein encoded by the Mdpcd1-307 cannot cause cell death when overexpressed (through the 35S promoter initiating gene expression), but when the Mdpcd1-307 is co-expressed with ARF1, it can cause cell death; similarly, when the Mdpcd1-307 is co-expressed with eEF1a, it can also cause cell death. It further shows that the interaction with ARF1 and eEF1a is the key factor to cause cell death, and the encoding protein that could not cause cell death can achieve the effect of causing cell death through co-expression.

3. The present invention obtains Mdpcd1-303 and its derived protein or homologous protein comprising the amino acid sequence shown in SEQ ID NO: 1 and/or SEQ ID NO: 29. These proteins have been proved to trigger the collapse of the cell membrane system and achieve the effect of destroying cells. They can significantly increase the apoptosis rate of tumor cells and significantly inhibit the growth of tumor tissue, thus having a therapeutic effect on tumors.

4. The amino acid sequence shown in SEQ ID NO: 1 and/or SEQ ID NO: 29 and the nucleotide encoding the amino acid sequence obtained in the present invention can also have therapeutic effects on tumors. The allele or core sequence of the Mdpcd1-303 also has similar functions.

5. Using any one of vectors that can guide the proper expression (including overexpression) of exogenous genes in tumor cells, the encoding nucleotide sequence of the Mdpcd1-303 protein fragment or its derived protein fragment Mdpcd1-18 or its derived protein fragment Mdpcd1-297 or its derived protein fragment Mdpcd1-307 or its homologous protein fragment Ptpcd1-296 provided in the present invention can be introduced into tumor cells, which can change the life process of the tumor cells, initiate the programmed death of the tumor cells, realize the inhibition and killing of tumor tissue, and enhance the immune function of the organism.

6. Because the protein comprising any that promotes the cell-destructive amino acid sequence and/or the protein encoded by nucleotide sequence of the present invention interacts with the GTP enzyme (GTPase) family (such as ARF1 or eEF1a), and the gene mutations in the enzyme family are related to multiple carcinogenesis, the present invention can also be used to prepare potential drugs for the treatment of cancers related to GTPase gene mutations.

The present invention will be further illustrated below with reference to the specific examples. It should be understood that these examples are only to illustrate the invention, not to limit the scope of the invention. The experimental methods with no specific conditions described in the following examples are generally performed under the conventional conditions (e.g., the conditions described by Sambrook et al., Molecular Cloning: A Laboratory Manual (New York: Cold Spring Harbor Laboratory Press, 1989), or according to the manufacturer's instructions. Unless indicated otherwise, all percentage and parts are calculated by weight.

Materials and Methods

Amino acid sequences and nucleotide sequences SEQ ID NO: 1 (Mdpcd1-9
amino acid sequence)
PTKQKHRSS
SEQ ID NO: 2 (Mdpcd1-303 amino acid sequence)
MCPTKQKHRSSVAEHAGKCSRSEDSTTSTAHWISESVHGGSLRHVDLTTGTNGWASPP
GDLFSLRAKNYLSKKQKGPAGDYLLQPCGTDWLRSSTKLENVLARPDNRVANALRKAQSQ
GRSLKSFIFAVNLQIPGKDQHSAVFYFATEDPIPAGSLLYRFVNGDDAFRNQRFKIVNRIVKGP
WIVEKTVGNYSACLLGKALTCNYHRGPNYLEIDVDIASFGIAKAILRLALRYVTSVTIDMGF
VVEAQAEDELPEKLVGAVRVCEMEMLSATVVEAPQTTVVARGLSFASKVNHHKSGDDDDD
D
SEQ ID NO: 3 (Mdpcd1-18 amino acid sequence)
MCPTKQKHRSSVAEHAGK
SEQ ID NO: 4 (Mdpcd1-297 amino acid sequence)
MCPTKQKHRSSVAEHAGKCSRSEDSTTSTAHWISESVHGGSLRHVDLTTGTNGWASPP
GDLFSLRAKNYLSKKQKGPAGDYLLQPCGTDWLRSSTKLENVLARPDNRVANALRKAQSQ
GRSLKSFIFAVNLQIPGKDQHSAVFYFATEDPIPAGSLLYRFVNGDDAFRNQRFKIVNRIVKGP
WIVEKTVGNYSACLLGKALTCNYHRGPNYLEIDVDIASFGIAKAILRLALRYVTSVTIDMGF
VVEAQAEDELPEKLVGAVRVCEMEMLSATVVEAPQTTVVARGLSFASKVNHHKSG
SEQ ID NO: 5 (Ptpcd1-296 amino acid sequence)
MHPTKQKHRSSGPESTTSRSSSTPGWITESINGGSLRHVDLHTGVNGWASPPGDLFSLR
SKNYFIKKQKSPSGDYLLSPAGMDWLKSSTKLDNVLARPDNRVANALKKAQSQNKSLKSFI
FAINLQVPGKDQHSAVFYFASEDPLPSDSLLYRFINGDDAFRNQRFKIVNRIEKGPWVVKKTV
GNYSACLLGKALNINYHRGGNYFEIDVDVGSSKIAAAILHLALGYTAHVTIDMGFVVEAQT
EEELPERLIGAIRVCQMEMSTARVVDSPSTGLARGSGFAKVEHHLSGDEEED
SEQ ID NO: 6 (Mdpcd1-303 nucleotide sequence)
ATGTGTCCGACGAAACAAAAGCACCGGAGCTCCGTCGCGGAACACGCCGGAAAAT
GCTCCAGATCGGAGGATTCTACAACCTCCACCGCCCACTGGATCTCCGAGTCCGTCCACG
GCGGATCCTTGCGCCACGTGGACCTCACCACCGGAACCAACGGCTGGGCGTCACCTCCC
GGGGACCTATTCTCGCTCCGCGCCAAGAACTACTTGTCGAAAAAACAAAAAGGCCCCGC
CGGCGACTACCTACTGCAGCCGTGCGGCACTGACTGGCTCAGATCCAGCACGAAACTCG
AGAACGTACTCGCTCGCCCCGATAATCGCGTGGCCAACGCGCTTCGGAAGGCACAGTCG
CAAGGGAGGTCCCTGAAGAGCTTCATTTTCGCCGTGAATCTCCAGATTCCCGGCAAGGA
CCAGCACAGCGCCGTTTTCTATTTCGCCACGGAGGATCCTATCCCTGCCGGCTCGCTTCTC
TACCGGTTCGTCAACGGCGACGACGCGTTCCGGAACCAGCGGTTCAAGATCGTGAACCG
GATCGTGAAAGGGCCGTGGATCGTCGAGAAGACGGTAGGGAATTACAGTGCGTGCTTGC
TGGGGAAGGCGCTGACGTGTAATTACCACAGAGGACCCAACTACCTCGAGATTGACGTC
GATATCGCGAGTTTTGGGATCGCGAAAGCGATTCTGCGACTTGCATTGAGGTACGTGACG
AGCGTGACGATCGACATGGGGTTTGTGGTGGAGGCGCAGGCGGAGGATGAGCTGCCTGA
GAAGTTAGTCGGCGCGGTTCGGGTATGCGAGATGGAGATGTTGTCTGCAACGGTCGTGG
AGGCGCCGCAGACGACGGTCGTAGCGCGGGGATTGAGTTTTGCGTCTAAGGTGAATCAT
CACAAGTCCGGCGACGACGACGACGACGATTGA
SEQ ID NO: 7 (Mdpcd1-18 nucleotide sequence)
ATGTGTCCGACGAAACAAAAGCACCGGAGCTCCGTCGCGGAACACGCCGGAAAAT
AG
SEQ ID NO: 8 (Mdpcd1-297 nucleotide sequence)
ATGTGTCCGACGAAACAAAAGCACCGGAGCTCCGTCGCGGAACACGCCGGAAAAT
GCTCCAGATCGGAGGATTCTACAACCTCCACCGCCCACTGGATCTCCGAGTCCGTCCACG
GCGGATCCTTGCGCCACGTGGACCTCACCACCGGAACCAACGGCTGGGCGTCACCTCCC
GGGGACCTATTCTCGCTCCGCGCCAAGAACTACTTGTCGAAAAAACAAAAAGGCCCCGC
CGGCGACTACCTACTGCAGCCGTGCGGCACTGACTGGCTCAGATCCAGCACGAAACTCG
AGAACGTACTCGCTCGCCCCGATAATCGCGTGGCCAACGCGCTTCGGAAGGCACAGTCG
CAAGGGAGGTCCCTGAAGAGCTTCATTTTCGCCGTGAATCTCCAGATTCCCGGCAAGGA
CCAGCACAGCGCCGTTTTCTATTTCGCCACGGAGGATCCTATCCCTGCCGGCTCGCTTCTC
TACCGGTTCGTCAACGGCGACGACGCGTTCCGGAACCAGCGGTTCAAGATCGTGAACCG
GATCGTGAAAGGGCCGTGGATCGTCGAGAAGACGGTAGGGAATTACAGTGCGTGCTTGC
TGGGGAAGGCGCTGACGTGTAATTACCACAGAGGACCCAACTACCTCGAGATTGACGTC
GATATCGCGAGTTTTGGGATCGCGAAAGCGATTCTGCGACTTGCATTGAGGTACGTGACG
AGCGTGACGATCGACATGGGGTTTGTGGTGGAGGCGCAGGCGGAGGATGAGCTGCCTGA
GAAGTTAGTCGGCGCGGTTCGGGTATGCGAGATGGAGATGTTGTCTGCAACGGTCGTGG
AGGCGCCGCAGACGACGGTCGTAGCGCGGGGATTGAGTTTTGCGTCTAAGGTGAATCAT
CACAAGTCCGGCTGA
SEQ ID NO: 9 (Ptpcd1-296 nucleotide sequence)
ATGCACCCAACCAAACAGAAACACCGGAGCTCCGGTCCGGAATCAACAACCTCCA
GATCATCATCAACACCCGGTTGGATCACCGAATCGATCAACGGTGGATCACTTCGCCACG
TGGACTTACACACTGGTGTTAACGGATGGGCGTCACCGCCAGGTGATCTTTTCTCTCTCC
GTTCAAAAAACTACTTCATCAAAAAACAGAAATCCCCCTCCGGCGACTACCTACTCTCAC
CCGCCGGTATGGACTGGCTCAAATCTAGCACAAAACTCGACAACGTACTCGCTCGTCCA
GATAATCGCGTGGCAAACGCCTTAAAGAAAGCACAATCCCAAAATAAGTCCCTCAAGTC
CTTCATTTTTGCTATCAACCTTCAAGTCCCCGGTAAAGACCAACACAGCGCCGTTTTCTAC
TTCGCATCGGAAGATCCTCTACCGTCCGATTCCCTCCTATATAGATTCATCAACGGCGATG
ATGCATTTCGGAATCAACGGTTCAAGATCGTGAACCGGATTGAAAAGGGTCCATGGGTG
GTGAAAAAAACGGTAGGAAATTACAGCGCGTGTTTATTAGGTAAAGCGTTAAATATCAAT
TACCATAGGGGTGGGAATTATTTCGAGATTGATGTTGATGTTGGCAGCTCGAAAATTGCTG
CGGCAATTTTGCATCTCGCATTGGGGTACACGGCGCATGTCACCATCGATATGGGGTTTGT
CGTGGAGGCGCAGACGGAGGAGGAGTTGCCGGAGAGGTTGATTGGGGCAATCAGGGTT
TGTCAGATGGAAATGTCGACGGCGCGTGTTGTTGATTCCCCGTCGACGGGTTTGGCGCGT
GGGTCGGGGTTTGCTAAGGTGGAGCATCATTTGTCAGGCGATGAAGAGGAGGATTGA
SEQ ID NO: 10 (P1 upstream primer nucleotide sequence)
ATGTGTCCAACAAAGCAAAAGC
SEQ ID NO: 11 (P1 downstream primer nucleotide sequence)
TCAATCGTCGTCGTCATCGTCG
SEQ ID NO: 12 (P2 upstream primer nucleotide sequence)
ATGTGTCCAACAAAGCAAAAGC
SEQ ID NO: 13 (P2 downstream primer nucleotide sequence)
TCAGCCGGACTTGTGATGATTCAC
SEQ ID NO: 14 (P3 upstream primer nucleotide sequence)
ATGCACCCAACCAAACAGAA
SEQ ID NO: 15 (P3 downstream primer nucleotide sequence)
TCAATCCTCCTCTTCATCGC
SEQ ID NO: 16 (P4 upstream primer nucleotide sequence)
CAGCCCATGGATGTGTCCAACAAAGCAAAAGC
SEQ ID NO: 17 (P4 downstream primer nucleotide sequence)
GAAGTCTAGATCAATCGTCGTCGTCATCGTCG
SEQ ID NO: 18 (P5 upstream primer nucleotide sequence)
CAGCCCATGGATGTGTCCAACAAAGCAAAAGC
SEQ ID NO: 19 (P5 downstream primer nucleotide sequence)
GAAGTCTAGATCAGCCGGACTTGTGATGATTCAC
SEQ ID NO: 20 (P6 upstream primer nucleotide sequence)
CAGCCCATGGATGCACCCAACCAAACAGAA
SEQ ID NO: 21 (P6 downstream primer nucleotide sequence)
GAAGTCTAGATCAATCCTCCTCTTCATCG
SEQ ID NO: 22 (P7 upstream primer nucleotide sequence)
AGCCACCATGGATGTGTCCAACAAAGCAAAAGC
SEQ ID NO: 23 (P7 downstream primer nucleotide sequence)
GTACCTCTAGATCAATCGTCGTCGTCATCGTCG
SEQ ID NO: 24 (P8 upstream primer nucleotide sequence)
AGCCACCATGGATGTGTCCAACAAAGCAAAAGC
SEQ ID NO: 25 (P8 downstream primer nucleotide sequence)
GTACCTCTAGATCAGCCGGACTTGTGATGAT
SEQ ID NO: 26 (P9 upstream primer nucleotide sequence)
AGCCACCATGCACCCAACCAAACAGAAACAC
SEQ ID NO: 27 (P9 downstream primer nucleotide sequence)
GTACCTCTAGATCAATCCTCCTCTTCATCGC
SEQ ID NO: 28 (Atpcd1 amino acid sequence)
MSPSKQRHRSSTGENKSKPVRSGSSSAISEWITESTNGGSLRRVDPDTGTDGWASPPGD
VFSLRSDSYLSKKQKTPAGDYLLSPAGMDWLKSSTKLENVLARPDNRVAHALRKAQSRGQS
LKSFIFAVNLQIPGKDHHSAVFYFATEEPIPSGSLLHRFINGDDAFRNQRFKIVNRIVKGPWVV
KAAVGNYSACLLGKALTCNYHRGPNYFEIDVDISSSAIATAILRLALGYVTSVTIDMGFLAEA
QTEEELPERLIGAVRVCQMEMSSAFVVDAPPPQQLPSQPCRTLSSAKVNHDEDED
SEQ ID NO: 29 (Mdpcd1-268 amino acid sequence)
WISESVHGGSLRHVDLTTGTNGWASPPGDLFSLRAKNYLSKKQKGPAGDYLLQPCGTD
WLRSSTKLENVLARPDNRVANALRKAQSQGRSLKSFIFAVNLQIPGKDQHSAVFYFATEDPIP
AGSLLYRFVNGDDAFRNQRFKIVNRIVKGPWIVEKTVGNYSACLLGKALTCNYHRGPNYLE
IDVDIASFGIAKAILRLALRYVTSVTIDMGFVVEAQAEDELPEKLVGAVRVCEMEMLSATVV
EAPQTTVVARGLSFASKVNHHKSG
SEQ ID NO: 30 (Mdpcd1-307 amino acid sequence)
MCPTKQKHRSSVAEHAGKCSRSEDSTTSTAHWISESVHGGSLRHVDLTTGTNGWASPP
GDLFSLRAKNYLSKKQKGPAGDYLLQPCGTDWLRSSTKLENVLARPDNRVANALRKAQSQ
GRSLKSFIFAVNLQIPGKDQHSAVFYFATEDPIPAGSLLYRFVNGDDAFRNQRFKIVNRIVKGP
WIVEKTVGNYSACLLGKALTCNYHRGPNYLEIDVDIASFGIAKAILRLALRYVTSVTIDMGF
VVEAQAEDELPEKLVGAVRVCEMEMLSATVVEAPQTTVVARGLSFASKVNHHKSGDDDDD
DDD
SEQ ID NO: 31 (Mdpcd1-307 nucleotide sequence)
ATGTGTCCGACGAAACAAAAGCACCGGAGCTCCGTCGCGGAACACGCCGGAAAAT
GCTCCAGATCGGAGGATTCTACAACCTCCACCGCCCACTGGATCTCCGAGTCCGTCCACG
GCGGATCCTTGCGCCACGTGGACCTCACCACCGGAACCAACGGCTGGGCGTCACCTCCC
GGGGACCTATTCTCGCTCCGCGCCAAGAACTACTTGTCGAAAAAACAAAAAGGCCCCGC
CGGCGACTACCTACTGCAGCCGTGCGGCACTGACTGGCTCAGATCCAGCACGAAACTCG
AGAACGTACTCGCTCGCCCCGATAATCGCGTGGCCAACGCGCTTCGGAAGGCACAGTCG
CAAGGGAGGTCCCTGAAGAGCTTCATTTTCGCCGTGAATCTCCAGATTCCCGGCAAGGA
CCAGCACAGCGCCGTTTTCTATTTCGCCACGGAGGATCCTATCCCTGCCGGCTCGCTTCTC
TACCGGTTCGTCAACGGCGACGACGCGTTCCGGAACCAGCGGTTCAAGATCGTGAACCG
GATCGTGAAAGGGCCGTGGATCGTCGAGAAGACGGTAGGGAATTACAGTGCGTGCTTGC
TGGGGAAGGCGCTGACGTGTAATTACCACAGAGGACCCAACTACCTCGAGATTGACGTC
GATATCGCGAGTTTTGGGATCGCGAAAGCGATTCTGCGACTTGCATTGAGGTACGTGACG
AGCGTGACGATCGACATGGGGTTTGTGGTGGAGGCGCAGGCGGAGGATGAGCTGCCTGA
GAAGTTAGTCGGCGCGGTTCGGGTATGCGAGATGGAGATGTTGTCTGCAACGGTCGTGG
AGGCGCCGCAGACGACGGTCGTAGCGCGGGGATTGAGTTTTGCGTCTAAGGTGAATCAT
CACAAGTCCGGCGACGACGACGACGACGACGACGATTGA

General Method

Obtaining the Encoding Nucleotide Sequence of the Target Protein Fragment by PCR Amplification

Referring to the published genome sequence of the target protein fragment, the corresponding upstream and downstream primers were designed with Macvector software. Subsequently, the total RNA of the corresponding experimental materials was extracted with the Plant Trizol kit of Invitrogen, the total RNA quality was identified by formaldehyde denaturing gel electrophoresis, and then the RNA content was measured on a spectrophotometer. A reverse transcription kit (Promega) was used for reverse transcription, a single-stranded cDNA was synthesized as a template, and the target fragment was amplified with the designed primers. The PCR reaction system was 25 μL, including 5 ng of template, 5 pmol of F primer and R primer respectively, 2.5 μL of 10×PCR buffer, 37.3 nmol of MgCl₂, 5 nmol of dNTP, and 0.5 U of rTaq polymerase. The amplification procedure was: pre-denaturation at 94° C. for 3 min; 94° C. for 20 s, 60° C. for 30 s, and 72° C. for 60 s, 30 cycles; reaction at 72° C. for 5 min.

Obtaining the Target Protein Fragment

The primers for introducing restriction endonuclease sites were designed, PCR amplification was carried out using the obtained target encoding nucleotide sequence as a template, and the PCR products were digested, purified and quantified, then cloned into the EcoR I and BamH I restriction sites of the expression vector PET-32a, and transformed into E. coli. Single colony was selected and shaken in 1 mL of LB (Amp 100 g/mL) overnight, and then transferred to 200 mL of fresh LB medium until the concentration of bacterial solution reached A600≈0.6; IPTG was added to the final concentration of 1.0 mM, and the induced expression was conducted at 37° C. for 3 hours. The bacterial solution was centrifuged at 12,000 g for 5 min, the precipitate was suspended in the extraction buffer (3 M NaCl, 1 mM PMSF, 50 mM pH8.0 phosphate buffer) and cells were disrupted by ultrasonication, then centrifuged at 12,000 g for 20 min, and the supernatant was collected. The Ni-Sepharose gel was balanced with 10 mM imidazole and 50 mM pH8.0 phosphate buffer; the cell lysate was added for incubation at room temperature for 20 min, and the Ni-Sepharose gel was washed 3 times using the balance buffer with a volume of 5 times the volume of the gel; then the Ni-Sepharose gel was eluted with 50 mM pH8.0 phosphate buffer containing 300 mM imidazole, and the eluent was collected as the purified Trx-expression protein. After desalting by dialysis, 0.1 mg of enterokinase per mg of protein sample was added into the purified expressed protein, and the histidine tag was removed after incubation at 25° C. for 2 hours in 40 mM succinic acid buffer (pH=5.6). The purified target protein fragment was obtained after overnight dialysis.

### Example 1 Obtaining the Encoding Nucleotide Sequence of the Mdpcd1-303 Protein Fragment

Referring to the published genome sequence of Malus, the P1 primers were designed with Macvector software. The P1 primers are shown as follows:

F-
(SEQ ID NO: 10)
5′-ATGTGTCCAACAAAGCAAAAGC-3′;
R-
(SEQ ID NO: 11)
5′-TCAATCGTCGTCGTCATCGTCG-3′

The total RNA of young leaves of Malus was extracted with the Plant Trizol kit (Invitrogen). According to the subsequent steps of obtaining the nucleotide sequence as described in the general method, 912 bp of the nucleotide sequence was obtained by PCR amplification. The PCR products were cloned into the pMD18-T vector, and the nucleotide sequence obtained by sequencing is shown in SEQ ID NO: 6. This sequence encodes 303 amino acids, and the amino acid sequence is shown in SEQ ID NO: 2.

### Example 2 Obtaining the Encoding Nucleotide Sequence of the Mdpcd1-18 Protein Fragment

The encoding nucleotide sequence (SEQ ID NO: 7) of the Mdpcd1-18 peptide was synthesized by gene synthesis technology. This sequence encodes 18 amino acids, and the amino acid sequence is shown in SEQ ID NO: 3.

### Example 3 Obtaining the Encoding Nucleotide Sequence of the Mdpcd1-297 Protein Fragment

Referring to the published genome sequence of Malus, the P2 primers were designed with Macvector software. The P2 primers are shown as follows:

F-
(SEQ ID NO: 12)
5′-ATGTGTCCAACAAAGCAAAAGC-3′;
R-
(SEQ ID NO: 13)
5′-TCAGCCGGACTTGTGATGATTCAC-3′

The total RNA of young leaves of Malus was extracted with the Plant Trizol kit (Invitrogen). According to the subsequent steps of obtaining the nucleotide sequence as described in the general method, 912 bp of the nucleotide sequence was obtained by PCR amplification. The PCR products were cloned into the pMD18-T vector, and the nucleotide sequence obtained by sequencing is shown in SEQ ID NO: 8. This sequence encodes 297 amino acids, and the amino acid sequence is shown in SEQ ID NO: 4.

### Example 4 Obtaining the Encoding Nucleotide Sequence of the Ptpcd1-296 Protein Fragment

Referring to the published genome sequence of Populus trichocarpa, the P3 primers were designed with Macvector software. The P3 primers are shown as follows:

F-
(SEQ ID NO: 14)
5′-ATGCACCCAACCAAACAGAA-3′;
R-
(SEQ ID NO: 15)
5′-TCAATCCTCCTCTTCATCGC-3′

The total RNA of young leaves of Populus trichocarpa was extracted with the Plant Trizol kit (Invitrogen). According to the subsequent steps of obtaining the nucleotide sequence as described in the general method, 891 bp of the nucleotide sequence was obtained by PCR amplification. The PCR products were cloned into the pMD18-T vector, and the sequence obtained by sequencing is shown in SEQ ID NO: 9. This sequence encodes 296 amino acids, and the amino acid sequence is shown in SEQ ID NO: 5.

### Example 5 Obtaining the Mdpcd1-303 Protein Fragment

The P4 primers for introducing endonuclease sites were designed, and the P4 primers are shown as follows:

F-
(SEQ ID NO: 16)
5′-CAGCCCATGGATGTGTCCAACAAAGCAAAAGC-3′;
R-
(SEQ ID NO: 17)
5′-GAAGTCTAGATCAATCGTCGTCGTCATCGTCG-3′

PCR amplification was carried out using the obtained Mdpcd1-303 encoding sequence (SEQ ID NO: 6) as a template. According to the subsequent steps of obtaining the target protein fragment as described in the general method, the purified protein fragment Mdpcd1-303 of Malus Mdpcd1 was obtained.

### Example 6 Obtaining the Mdpcd1-18 Protein Fragment

The Mdpcd1-18 encoding sequence (SEQ ID NO: 7), which was connected to EcoR I and BamH I endonuclease sites at both ends, was obtained through gene synthesis, the gene synthesis products were subjected to the subsequent steps of obtaining the target protein fragment as described in the general method, and the purified protein fragment Mdpcd1-18 was obtained.

Since the protein fragment Mdpcd1-18 is a small molecule peptide, it can also be directly synthesized by peptide synthesis technology.

### Example 7 Obtaining the Mdpcd1-297 Protein Fragment

The P5 primers for introducing endonuclease sites were designed, and the P5 primers are shown as follows:

F-
(SEQ ID NO: 18)
5′-CAGCCCATGGATGTGTCCAACAAAGCAAAAGC-3′;
R-
(SEQ ID NO: 19)
5′-GAAGTCTAGATCAGCCGGACTTGTGATGATTCAC-3′

PCR amplification was carried out using the obtained Mdpcd1 encoding sequence (SEQ ID NO: 8) as a template. According to the subsequent steps of obtaining the target protein fragment as described in the general method, the purified protein fragment Mdpcd1-297 was obtained.

### Example 8 Obtaining the Ptpcd1-296 Protein Fragment

The P6 primers for introducing endonuclease sites were designed, and the P6 primers are shown as follows:

F-
(SEQ ID NO: 20)
5′-CAGCCCATGGATGCACCCAACCAAACAGAA-3′;
R-
(SEQ ID NO: 21)
5′-GAAGTCTAGATCAATCCTCCTCTTCATCG-3′

PCR amplification was carried out using the obtained Mdpcd nucleotide 1 sequence (SEQ ID NO: 9) as a template. According to the subsequent steps of obtaining the target protein fragment as described in the general method, the purified protein fragment Ptpcd1-296 was obtained.

For each protein obtained above, SEQ ID NO: 1 and/or SEQ ID NO: 29 are conservative functional regions.

### Example 9 Cell Destruction Test of Each Protein Obtained Above

In this example, the encoding sequences of the proteins in the above examples were respectively constructed into transient expression vectors. The tobacco leaves were subjected to 72-hour immersion test with the transient expression vectors, and in the meantime the empty vector was used as the control group.

In addition, the transient expression vector which comprised the Atpcd1 protein encoding sequence of Arabidopsis homologous to the Mdpcd1-303 protein was also used for the same immersion test. The amino acid sequence of the Atpcd1 protein is shown in SEQ ID NO: 28, and this sequence does not have the conservative sequences (SEQ ID NO: 1 and/or SEQ ID NO: 29) of the proteins obtained in the above examples.

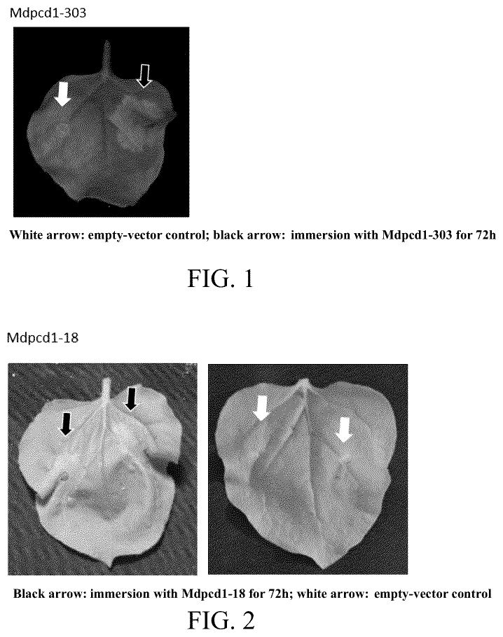

FIG. 1 shows the results of the immersion test with the transient expression vector involved in Example 9, which comprises the encoding sequence of the Mdpcd1-303 protein fragment;

FIG. 2 shows the results of the immersion test with the transient expression vector involved in Example 9, which comprises the encoding sequence of the Mdpcd1-18 protein fragment;

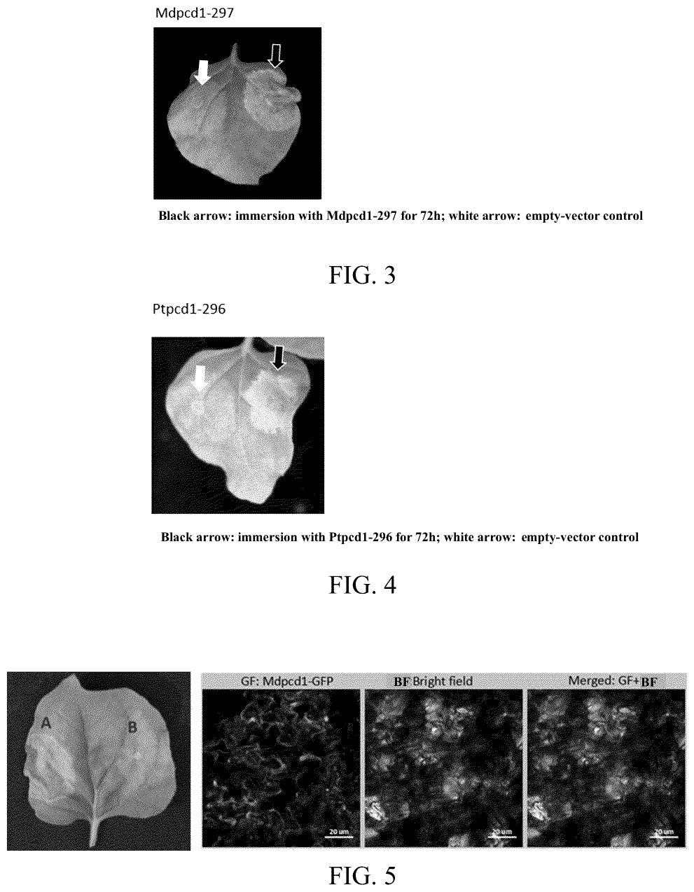

FIG. 3 shows the results of the immersion test with the transient expression vector involved in Example 9, which comprises the encoding sequence of the Mdpcd1-297 protein fragment;

FIG. 4 shows the results of the immersion test with the transient expression vector involved in Example 9, which comprises the encoding sequence of the Ptpcd1-296 protein fragment.

As shown in FIGS. 1-4, compared with the empty vector control, the tobacco leaves were damaged after immersion through the immersion test.

FIG. 5 shows the changes of tobacco leaf cells after the immersion test with the transient expression vector involved in Example 9, which comprises the encoding sequence of the Mdpcd1-303 protein fragment.

As shown in FIG. 5, the damaged tobacco leaf after the immersion test with the transient expression vector comprising the encoding sequence of the Mdpcd1-303 protein fragment was placed in the cell field of vision (A region), and the Mdpcd1-303-GFP fusion protein located on the cell membrane diffused and disintegrated with the membranolysis (in FIG. 5, GF: GFP fluorescence position is the cell membrane position, GFP fluorescence signal is the location information of the Mdpcd1-303; BF: the cell membrane disintegration image under white light; Merged: the overlapping of BF and GF, which can indicate that the Mdpcd1-303-GFP is located on the cell membrane, and it diffuses and disintegrates with the membranolysis). It showed that the Mdpcd1-303 protein fragment can destroy cells. According to FIGS. 2-5, it is also speculated that the derived or homologous fragments of the Mdpcd1-303 protein fragment can also destroy cells.

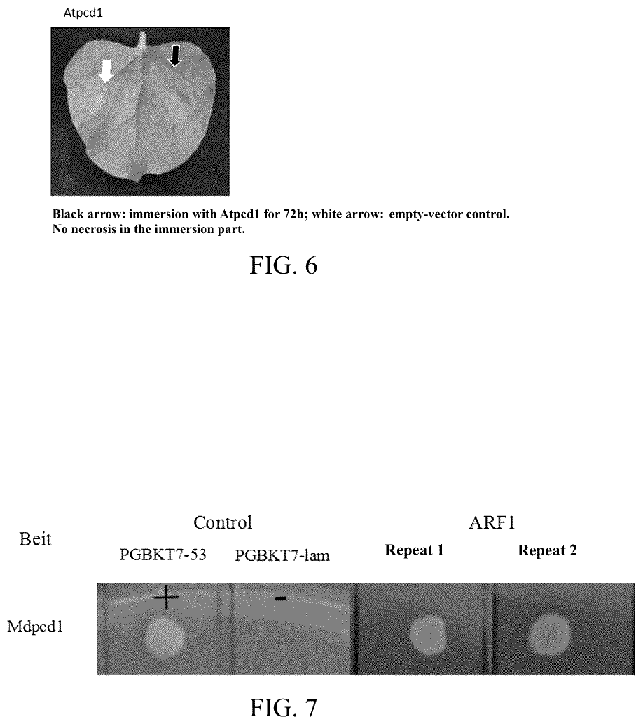

FIG. 6 shows the results of the immersion test with the transient expression vector involved in Example 9, which comprises the encoding sequence of the Atpcd1 protein.

As shown in FIG. 6, after the immersion test with the transient expression vector comprising the encoding sequence of the Atpcd1 protein, there was no necrosis in the tobacco leaves, so it was further confirmed that the amino acid sequence shown in SEQ ID NO: 1 and/or SEQ ID NO: 29 were conservative functional regions causing cell damage. Since the amino acid sequences shown in SEQ ID NO: 1 and/or SEQ ID NO: 29 have biological universality, it can be inferred that an amino acid sequence comprising these sequences can also cause damage to animal cells.

### Example 10 Analysis of Protein Interaction Between the Mdpcd1-303 Protein and the ARF1 by Yeast-Two Hybrid

FIG. 7 shows the identification of protein interaction between the Mdpcd1-303 protein involved in Example 10 and the ARF1 by yeast-two hybrid (QDO medium).

The Mdpcd1 bait plasmid and AFR1 recombinant prey plasmid were constructed, and a large number of the constructed bait plasmid and prey plasmid were extracted and detected by agarose gel electrophoresis. The ARF1 protein has cross-species sequence and function conservation. The ARF1 sequence of Malus (LOC103404322) used in the experiments was from the public database and was commonly known and used. The obtained bait plasmid was tested for toxicity and self-activation. The bait plasmid and the prey empty vector were co-transformed into NMY51 receptive cells. If the colony can grow on the DDO spread plate, it means that the recombinant bait plasmid has been successfully transferred into the host bacteria and is non-toxic to the host bacteria. If the colony cannot grow on the TDO and QDO spread plates, it means that the bait protein cannot activate the expression of the reporter gene. The bait plasmid and the POST-NubaI plasmid were co-transformed into NMY51 receptive cells, and the colony could grow on the DDO spread plate, which showed that the co-transformation was successful. The colony could grow on the TDO and QDO spread plates, which indicated that the reporter genes HIS and ADE2 were activated, the reading frame constructed by bait was correct, and the ubiquitin experimental system was feasible. The prey plasmid and bait plasmid were co-transformed into yeast cells, and the results of control and functional verification were in line with expectations, indicating that the system could be used for two-hybrid verification; the host bacteria transformed by the prey plasmid and bait plasmid could grow on the DDO spread plate, indicating that it can grow on the SD-TLHA spread plate, and there was interaction between them (FIG. 7).

This example showed that the protein fragment comprising the amino acid sequence shown in SEQ ID NO: 1 and/or SEQ ID NO: 29 might cause damage to cells through the interaction with ARF1.

### Example 11 Application of the Encoding Nucleotide Sequence of the Mdpcd1-303 Protein Fragment

The treatment of the recombinant adenovirus Mdpcd1-303 on tumor-bearing mouse models with hepatocellular carcinoma was taken as an example.

Since the target of the Mdpcd303, ARF1, is fundamental and conservative in animals and plants, the programmed cell death mediated by the Mdpcd1-303 and other protein fragments comprising the amino acid sequence shown in SEQ ID NO: 1 and/or SEQ ID NO: 29 is also conservative in animals and plants, and the proliferation inhibition, killing and death of tumors and other pathological cells mediated by these proteins have the characteristic of universality. This example is one of many similar examples of functional application of the Mdpcd1-303 protein fragments comprising the amino acid sequence SEQ ID NO: 1 and/or SEQ ID NO: 29.

Construction of the recombinant adenovirus Mdpcd1-303 and cell packaging were completed by commercial technical services provided by biotechnology companies.

Construction of the transfer plasmid pShuttle-CMV-Mdpcd1-303:

The Mdpcd1-cDNA template was amplified by PCR, and the Mdpcd1-303 upstream primer P7 is shown as follows:

(SEQ ID NO: 22)
5′-AGCCACCATGGATGTGTCCAACAAAGCAAAAGC-3′;

Downstream primer:

(SEQ ID NO: 23)
5′-GTACCTCTAGATCAATCGTCGTCGTCATCGTCG-3′.

The expected amplified fragment was 940 bp, and the PCR reaction conditions were: 94° C. for 3 min; 94° C. for 20 s, 58° C. for 30 s, and 72° C. for 1 min, 30 cycles; 72° C. for 7 min. 1% agarose gel electrophoresis was used for nucleic acid separation, and the gel was then cut and recycled. The transfer plasmid pShuttle-CMV and the target gene Mdpcd1 were digested and purified by Kpn I and Xba I, and the two were linked by a T4 DNA ligase. The Mdpcd1-303 was directionally cloned into the transfer plasmid pShuttle-CMV, and then digested and sequenced to identify the accuracy of the cloning. Construction of the recombinant adenovirus and the packaging, amplification, purification and identification of the virus in HEK293 cells: the transfer plasmid pShuttle-CMV-Mdpcd1-303 confirmed to be cloned successfully after identification and the skeleton plasmid pAdc68 were digested and linearized by PI-sce I and Iceu I. After being correctly identified by electrophoresis, the gel was cut and recovered. Then the fragments were connected at 16° C. overnight, and converted into E. coli stab-2. LB plate (ampicillin) was made for screening, clones were selected for shaking and plasmid extraction. The plasmid was digested by Bagl II, Xhol I, and Mun I, and the result of sequencing was correct. The positive recombinant clone pShuttle-CMV-Mdpcd1-303-pAdc68 was successfully constructed. When HEK293 cells grew to about 80% fusion rate, the pShuttle-CMV-Mdpcd1-303-pAdc68 was digested and linearized by Pac I. The HEK293 cells were transfected with X-treme method, and the virus was packaged in the HEK293 cells, and cultured for 12 days. Under the light microscope, the cells became round, shed off from the bottom wall of the culture bottle, and the nucleus occupied most of the cell volume, which indicated that the cytopathic effect (CPE) occurred. The cells were collected, and centrifuged at 3500 r/min for 5 minutes. The supernatant was removed and the cells were resuspended in antibiotic-free, serum-free DMEM. Then the cells were repeatedly freeze-thawed at 37° C. and −80° C. for three times. The HEK293 cells were reinfected and expanded. After repeated amplification for three to four generations, CsCl gradient centrifugation for purification was performed to obtain the adenovirus vector with the Mdpcd1-303 gene (named Adc68-Mdpcd1-303), which was stored at −80° C. for future use.

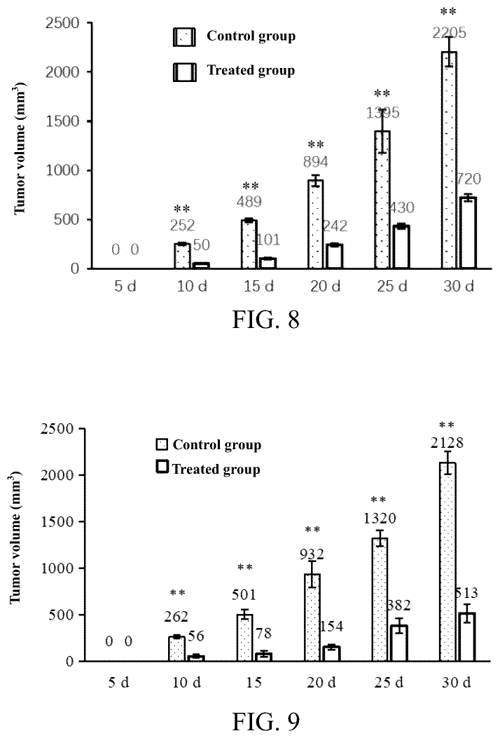

FIG. 8 shows the subcutaneous tumor growth curves of mice treated with Adc68-Mdpcd1-303 injection and tumor-bearing mice as control group in Example 11. **P<0.01.

Thirty mice with successful tumorigenesis experiment of hepatocellular carcinoma were randomly selected and randomly divided into two groups, receiving intratumoral injection respectively: the experimental group with Adc68-Mdpcd1-303 (1×10⁹ PFU); the blank control group with 100 μL of PBS; the tumor volume of mice was measured every 3 days and the survival of mice was observed at any time. The measurement formula of the tumor volume was: volume=length/2×width². Compared with the PBS blank control group, Adc68-Mdpcd1-303 could significantly inhibit tumor growth (FIG. 8). In terms of survival time, the death of nude mice in Adc68-Mdpcd1-303 treatment group occurred after 40 days of treatment, however, all mice in the control group died within 40 days. In addition, the average life span of nude mice in Adc68-Mdpcd1-303 group was significantly prolonged, and about half of them survived for more than 55 days. The recombinant virus Adc68-Mdpcd1-303 had obvious protective effects on the growth of nude mouse xenograft models.

### Example 12 Application of the Encoding Sequence of the Mdpcd1-18 Protein Fragment

The treatment of the recombinant adenovirus Mdpcd1-18 on tumor-bearing mouse models with hepatocellular carcinoma was taken as an example.

Construction of the recombinant adenovirus Mdpcd1-18 and cell packaging were completed according to Example 11. The encoding nucleotide sequence of the Mdpcd1-18 was synthesized artificially, and Kpn I and Xba I restriction sites were added at both ends of the encoding nucleotide sequence during synthesis.

FIG. 9 shows the subcutaneous tumor growth curves of mice treated with Adc68-Mdpcd1-18 injection and tumor-bearing mice as control group in Example 12. **P<0.01.

The inhibition experiment of hepatocellular carcinoma in mice was carried out with reference to Example 11. Compared with the PBS blank control group, Adc68-Mdpcd1-18 could significantly inhibit tumor growth (FIG. 9). In terms of survival time, the death of nude mice in Adc68-Mdpcd1-18 treatment group occurred after 40 days of treatment, however, all mice in the control group died within 40 days. In addition, the average life span of nude mice in Adc68-Mdpcd1-18 group was significantly prolonged, and about half of them survived for more than 60 days. The recombinant virus Adc68-Mdpcd1-18 had obvious protective effect on the growth of nude mouse xenograft models.

### Example 13 Application of the Encoding Sequence of the Mdpcd1-297 Protein Fragment

The treatment of the recombinant adenovirus Mdpcd1-297 on tumor-bearing mouse models with hepatocellular carcinoma was taken as an example.

Construction of the recombinant adenovirus Mdpcd1-297 and cell packaging were completed according to Example 11.

P8 PCR primers for construction of the transfer plasmid pShuttle-CMV-Mdpcd1-297:

Upstream primer:
(SEQ ID NO: 24)
5′-AGCCACCATGGATGTGTCCAACAAAGCAAAAGC-3′;
Downstream primer:
(SEQ ID NO: 25)
5′-GTACCTCTAGATCAGCCGGACTTGTGATGAT-3′

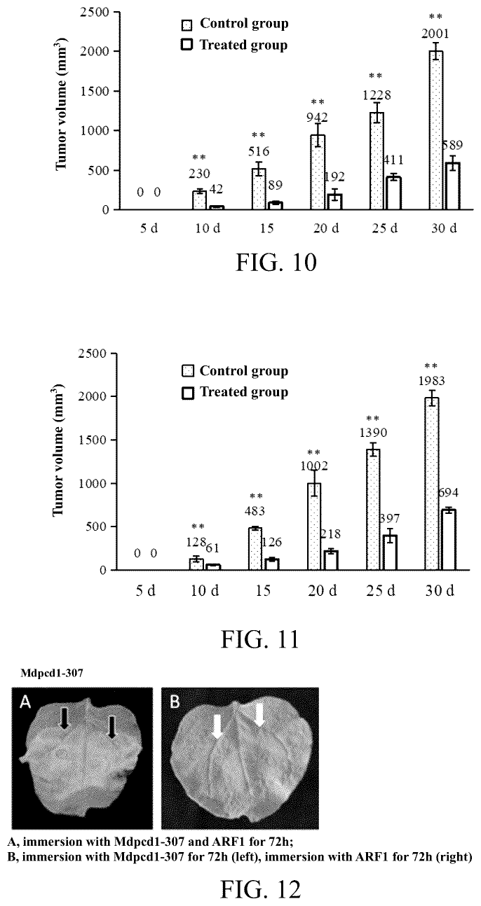

FIG. 10 shows the subcutaneous tumor growth curves of mice treated with Adc68-Mdpcd1-297 injection and tumor-bearing mice as control group in Example 13. **P<0.01.

The inhibition experiment of hepatocellular carcinoma in mice was carried out with reference to Example 11. Compared with the PBS blank control group, Adc68-Mdpcd1-297 could significantly inhibit tumor growth (FIG. 10). In terms of survival time, the death of nude mice in Adc68-Mdpcd1-297 treatment group occurred after 40 days of treatment, however, all mice in the control group died within 40 days. In addition, the average life span of nude mice in Adc68-Mdpcd1-297 group was significantly prolonged, and about half of them survived for more than 58 days. The recombinant virus Adc68-Mdpcd1-297 had obvious protective effect on the growth of nude mouse xenograft models.

### Example 14 Application of the Encoding Sequence of the Ptpcd1-296 Protein Fragment

The treatment of the recombinant adenovirus Ptpcd1-296 on tumor-bearing mouse models with hepatocellular carcinoma was taken as an example.

Construction of the recombinant adenovirus Ptpcd1-296 and cell packaging were completed according to Example 11.

P9 PCR primers for construction of the transfer plasmid pShuttle-CMV-Ptpcd1-296:

Upstream primer:
(SEQ ID NO: 26)
5′-AGCCACCATGCACCCAACCAAACAGAAACAC-3′;
Downstream primer:
(SEQ ID NO: 27)
5′-GTACCTCTAGATCAATCCTCCTCTTCATCGC-3′

FIG. 11 shows the subcutaneous tumor growth curves of mice treated with Adc68-Ptpcd1-296 injection and tumor-bearing mice as control group in Example 14. **P<0.01.

The inhibition experiment of hepatocellular carcinoma in mice was carried out with reference to Example 11. Compared with the PBS blank control group, Adc68-Ptpcd1-296 could significantly inhibit tumor growth (FIG. 11). In terms of survival time, the death of nude mice in Adc68-Ptpcd1-296 treatment group occurred after 40 days of treatment, however, all mice in the control group died within 40 days. In addition, the average life span of nude mice in Adc68-Ptpcd1-296 group was significantly prolonged, and about half of them survived for more than 59 days. The recombinant virus Adc68-Ptpcd1-296 had obvious protective effect on the growth of nude mouse xenograft models.

### Example 15 Obtaining the Encoding Sequence of the Mdpcd1-307 Protein Fragment

The encoding nucleotide sequence (SEQ ID NO: 31) of the Mdpcd1-307 peptide was synthesized by gene synthesis technology. This sequence encodes 307 amino acids, and the amino acid sequence is shown in SEQ ID NO: 30.

### Example 16 Obtaining the Mdpcd1-307 Protein Fragment

The Mdpcd1-307 encoding sequence (SEQ ID NO: 31), which was connected to EcoR I and BamH I endonuclease sites at both ends, was obtained through gene synthesis, and the gene synthesis products were subjected to the subsequent steps of obtaining the target protein fragment as described in the general method to obtain the purified protein fragment Mdpcd1-307, in which SEQ ID NO: 1 and/or SEQ ID NO: 29 were conservative functional regions.

### Example 17 Test in which the Co-Expression with ARF1 Enabled the Mdpcd1-307 Protein to Destroy Cells

In this example, the encoding sequence of the Mdpcd1-307 protein of the above example and the encoding sequence of ARF1 (LOC103404322) were respectively constructed into transient expression vectors. The same tobacco leaf was subjected to 72-hour immersion test with both the transient expression vectors, and in the meantime the transient expression vectors were used separately for immersion test as the control groups.

As shown in FIG. 12, the overexpression of ARF1 and Mdpcd1-307 initiated cell necrosis.

### Example 18 Application of the Encoding Nucleotide Sequence of the Mdpcd1-307 Protein Fragment

The inhibition of SMMC-7721 cell viability by transfection of the recombinant plasmid Mdpcd1-307 was taken as an example.

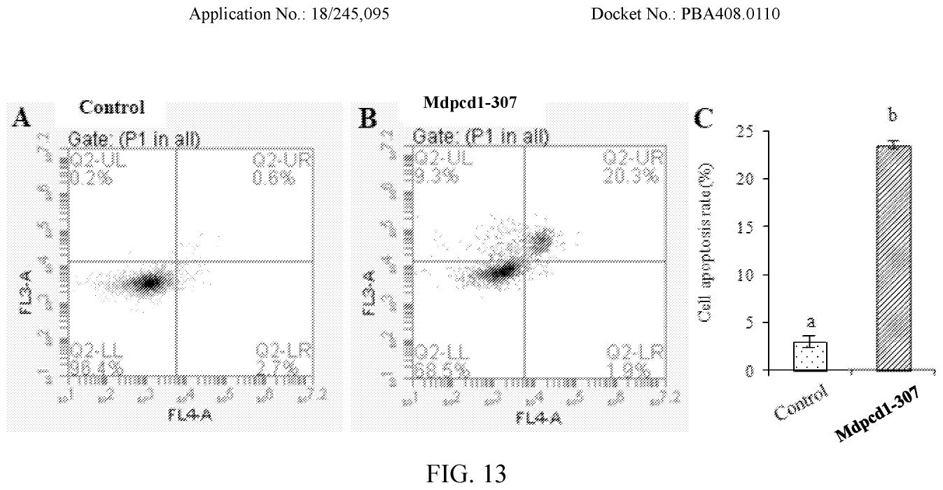

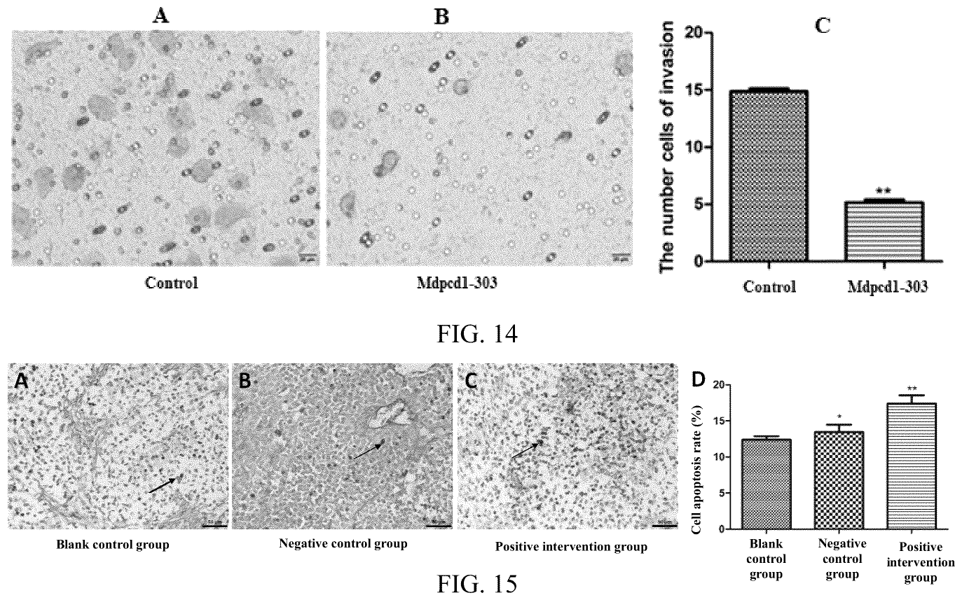

The Mdpcd1-307 encoding sequence which was connected to EcoR I and Kpn I endonuclease sites at both ends, was obtained through gene synthesis. The eukaryotic expression vector pEGFP-N1 and the target gene Mdpcd1-307 were digested and purified by EcoR I and Kpn I, and the two were linked by a T4 DNA ligase. The Mdpcd1-307 was directionally cloned into the eukaryotic expression vector pEGFP-N1, and then digested and sequenced to identify the accuracy of the cloning. The human hepatoma cells SMMC-7721 were cultured and placed into the culture plates. When the cell fusion rate reached 60%, the cells were divided into the empty plasmid group and the target plasmid group for transfection. Each group had 3 repeat wells, and each group was added with transfection reagent and culture medium without penicillin. After transfection, the cells were placed in an incubator for interference for 8 hours, and then the complete culture medium containing penicillin was used. The cells were collected for detection after 48 hours. The expression of the target gene was detected by qPCR. The apoptosis of SMMC-7721 cells was detected and analyzed by flow cytometry, and Mdpcd1-307 could significantly increase the apoptosis rate of SMMC-7721 cells (FIG. 13). Cell invasion analysis: the cell with Matrigel in the upper chamber of the 24-well plate was incubated in the cell incubator for more than 6 hours, and was taken out under sterile conditions. 100 μL of preheated serum-free medium was added into the upper chamber, kept at room temperature for 30 minutes, making the Matrigel hydrated, and then removed the remaining medium; after 48 hours of transfection and culture, the cells were digested with 0.25% Trypsin+0.02% EDTA and centrifuged, resuspended with 2% serum medium, and counted. The cells were divided into groups at a density of 10×10⁴/well and plated into the upper chamber of a 24-well plate. In the lower chamber, 10% serum medium was added, and the cells were incubated overnight in an incubator at 37° C. with 5% CO₂. After 12 h of culture, the plate was washed three times with 1×PBS, fixed with 4% paraformaldehyde at room temperature for 15 min, and then washed three times with 1×PBS again. The cells in the upper chamber were wiped off with a cotton swab, and the crystal violet was added for staining for 15 min. The plate was washed three times with 1×PBS, dried at room temperature, and photographed under a microscope. The experiment was repeated three times. Mdpcd1-307 could significantly reduce the invasive ability of SMMC-7721 cells (FIG. 14).

### Example 19 Application of the Encoding Nucleotide Sequence of the Mdpcd1-307 Protein Fragment

The antitumor effect of the Mdpcd1-307 on nude mice inoculated with SMMC-7721 hepatoma cells was taken as an example.

Construction of the Mdpcd1-307 recombinant adenovirus and cell packaging were completed by commercial technical services provided by biotechnology companies.

The transfer plasmid pShuttle-CMV-Mdpcd1-307 was constructed according to Example 11.

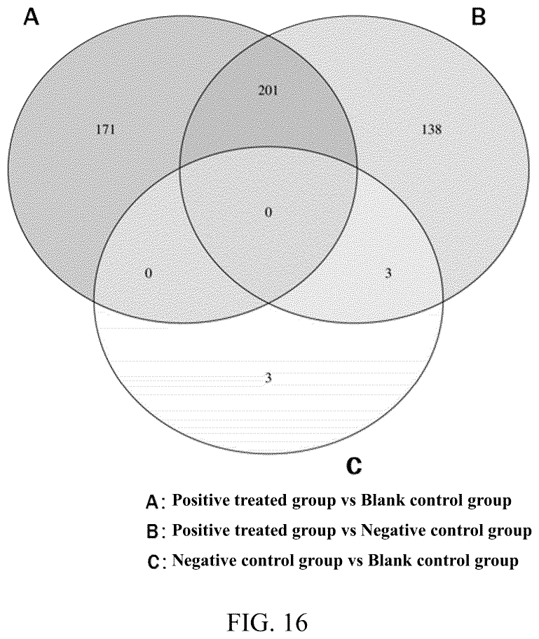

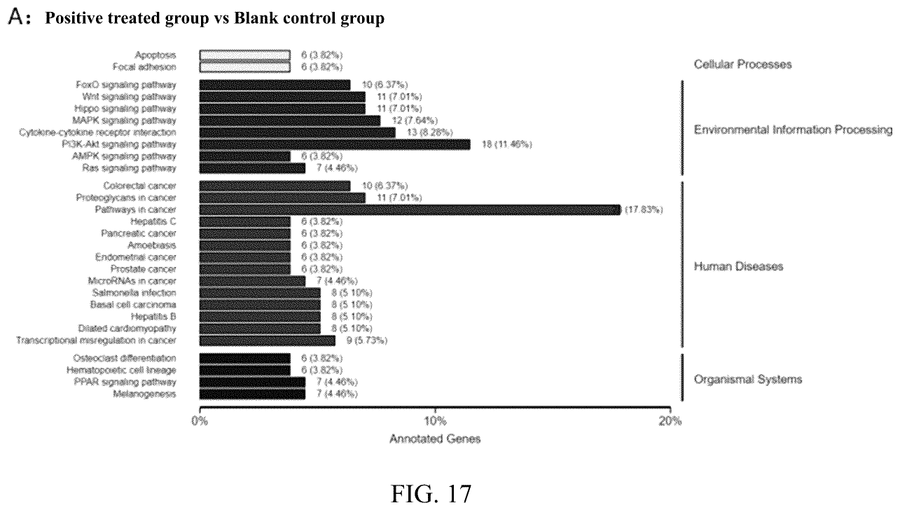

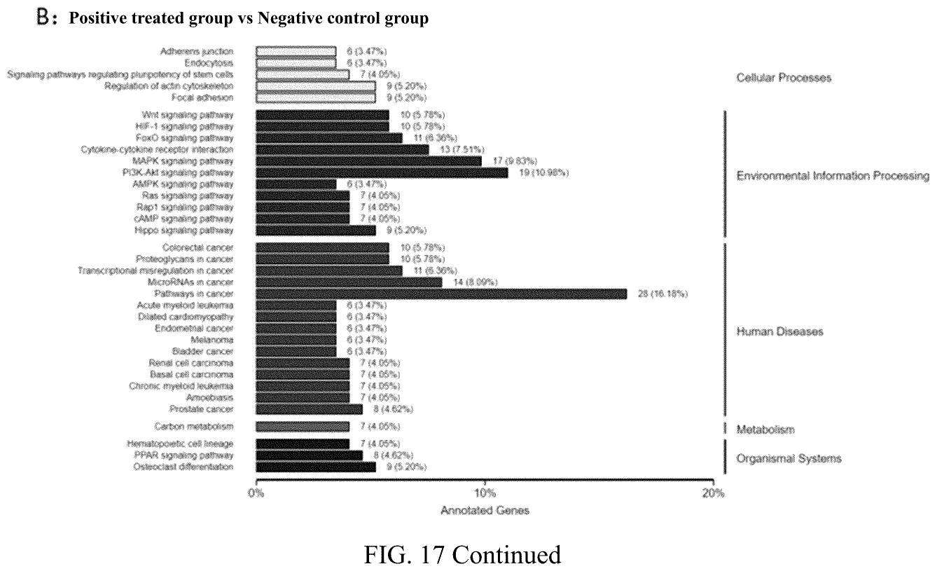

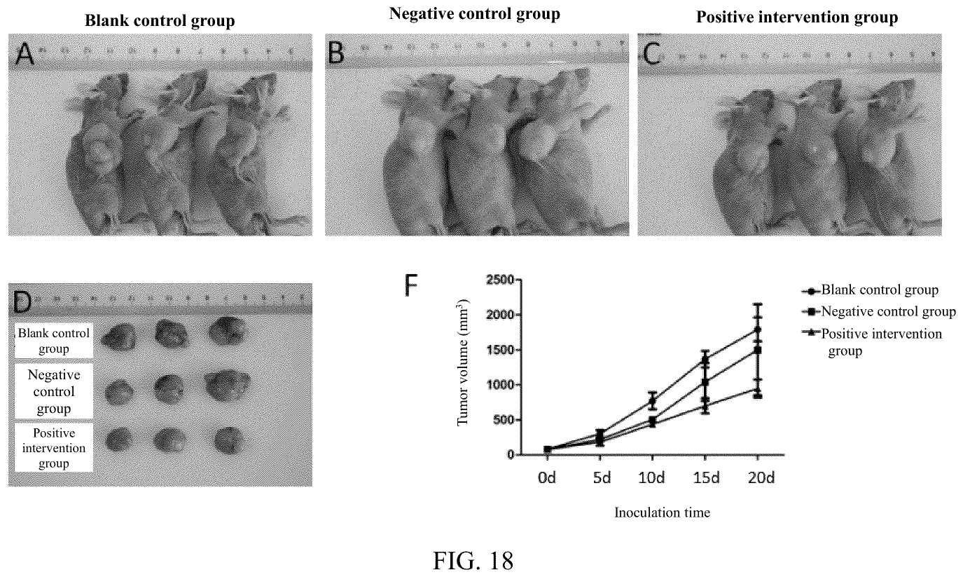

Nine BABL/c nude mice were randomly divided into blank control group, negative control group and positive intervention group, with 3 in each group. The subcutaneous tumorigenesis model was constructed with the amount of 1×10⁸ SMMC-7721 hepatoma cells/mouse. After the third day of cancer cell inoculation, the blank control group was inoculated with 100 μL of normal saline at the tumor site, the negative control group was inoculated with 100 μL of 10⁸ U/mL empty vector, and the positive intervention group was injected with 100 μL of corresponding 10⁸ PFU/mL adenovirus. After the intervention, the animals in each group were continuously observed and the changes of food intake and body weight were recorded regularly. When visible tumors were observed, the growth of tumor-bearing mice and the size of the subcutaneous xenograft tumor were recorded every 5 days to draw the growth curve. The experiment was terminated 20 days after inoculation, and the sample was fixed for standby. The preparation of tumor tissue sections and Tunel staining were carried out according to the routine experimental process. The results showed that the expression of the Mdpcd1-307 made the tumor cell apoptosis rate of SMMC-7721 hepatoma cells in nude mice significantly higher than that in the blank control group or negative control group (FIG. 15). With reference to the standard molecular experimental process, RNA of each sample was extracted, library construction and high-throughput sequencing were carried out, and the sequencing data were subject to quality control, reference gene anchoring and gene expression abundance calculation. With the blank control group or negative control group as reference, the effect of the Mdpcd1-307 expression on the tumor genes of SMMC-7721 hepatoma cell nude mice was analyzed, and the functional annotation and metabolic pathway enrichment analysis of the differentially expressed genes were carried out. Compared with the blank control group or negative control group, the Mdpcd1-307 expression changed the expression of many genes in SMMC-7721 hepatoma cell nude mice (FIG. 16). These genes were enriched in different metabolic or regulatory pathways related to tumor development (FIG. 17). Mdpcd1-307 significantly inhibited the tumor development of SMMC-7721 hepatoma cells in nude mice (FIG. 18).

### Example 20 Test in which the Interference on ARF1 Expression Inhibited the Destruction of Cells by the Mdpcd1-303 Protein

In this example, the transient expression vector which comprised the Mdpcd1-303 encoding sequence of the protein in the above example, and the ARF1 (LOC103404322)—RNAi interference expression vector were inoculated into the same tobacco leaf. The ARF1—RNAi interference expression vector was inoculated 48 hours earlier than the Mdpcd1-303 transient expression vector, and the co-immersion time was 72 hours. In the meantime, the vectors were used separately for immersion test as the control groups.

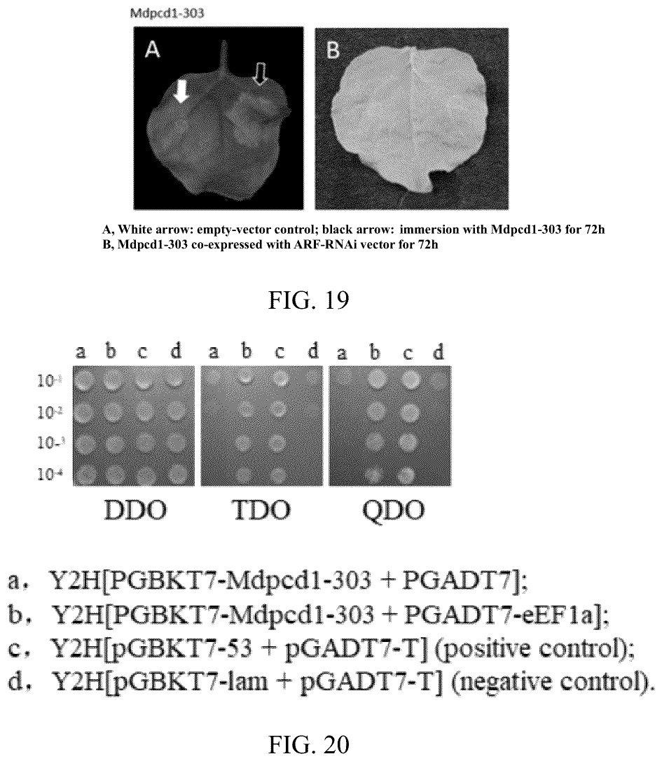

As shown in FIG. 19, the interference on ARF1 expression inhibited the cell necrosis caused by the overexpression of the Mdpcd1-303.

### Example 21 Interaction Between the Mdpcd1-303 Protein and eEF1a

Eukaryotic translation elongation factor eEF1a is highly expressed and plays a key role in tumors (including breast cancer, ovarian cancer and lung cancer, etc.) and many human diseases (Abbas et al., Front. Oncol., 7 Apr. 2015|https://doi.org/10.3389/fonc.2015.00075). Therefore, at this time, eEF1a can be used as a target to initiate cell destruction through the interaction with eEF1a, thereby achieving therapeutic effects on cancer and other diseases.

FIG. 20 shows the identification of protein interaction between the Mdpcd1-303 protein involved in Example 21 and the eEF1a by yeast-two hybrid.

### Example 22 Test in which the Interference on eEF1a Expression Inhibited the Destruction of Cells by the Mdpcd1-303 Protein

In this example, the transient expression vector which comprised the Mdpcd1-303 encoding sequence of the protein in the above example, and the eEF1a (LOC103447856)—RNAi interference expression vector were inoculated into the same tobacco leaf. The eEF1a—RNAi interference expression vector was inoculated 48 hours earlier than the Mdpcd1-303 transient expression vector, and the co-immersion time was 72 hours. In the meantime, the vectors were used separately for immersion test as the control groups.

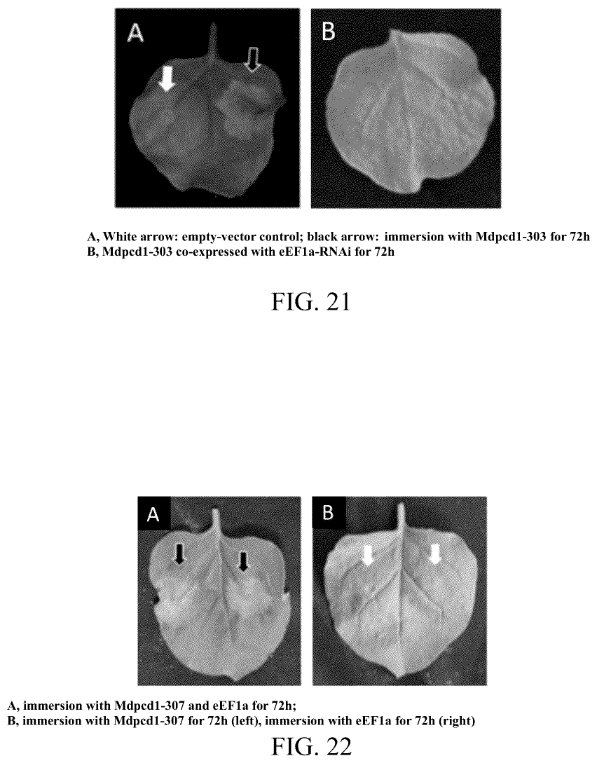

As shown in FIG. 21, the interference on eEF1a expression inhibited the cell necrosis caused by the overexpression of the Mdpcd1-303.

### Example 23 Test in which the Co-Expression with eEF1a Enabled the Mdpcd1-307 Protein to Destroy Cells

In this example, the encoding sequence of the Mdpcd1-307 protein of the above example and the encoding sequence of eEF1a (LOC103447856) were respectively constructed into transient expression vectors. The same tobacco leaf was subjected to 72-hour immersion test with both the transient expression vectors, and in the meantime the transient expression vectors were used separately for immersion test as the control groups.

As shown in FIG. 22, the overexpression of eEF1a and Mdpcd1-307 initiated the cell necrosis.

Discussion

The above embodiments illustrate that the amino acid sequence comprising SEQ ID NO: 1 and/or SEQ ID NO: 29, or the nucleotide sequence which can encode the amino acid sequence comprising SEQ ID NO: 1 and/or SEQ ID NO: 29 involved herein, or the vector comprising the nucleotide sequence, can be used in cell destruction or tumor treatment, in the manufacture of a composition for destroying cells, and in the manufacture of a medicament for use in tumor treatment, so as to prepare and obtain a composition for destroying cells which comprises the amino acid sequence comprising SEQ ID NO: 1 and/or SEQ ID NO: 29, or the nucleotide sequence encoding the amino acid sequence comprising SEQ ID NO: 1 and/or SEQ ID NO: 29 involved herein, or the vector comprising the nucleotide sequence, or a medicament for use in tumor treatment which comprises the amino acid sequence comprising SEQ ID NO: 1 and/or SEQ ID NO: 29, or the nucleotide sequence encoding the amino acid sequence comprising SEQ ID NO: 1 and/or SEQ ID NO: 29 involved herein, or the vector comprising the nucleotide sequence. ARF1 and eEF1a belong to GTPase, and gene mutations in this enzyme family are related to multiple carcinogenesis. The amino acid sequence comprising SEQ ID NO: 1 and/or SEQ ID NO: 29, or the nucleotide sequence which can encode the amino acid sequence comprising SEQ ID NO: 1 and/or SEQ ID NO: 29 involved herein, or the vector comprising the nucleotide sequence, is a potential drug for the treatment of cancers related to GTPase gene mutations.

All documents mentioned in the present invention are incorporated herein by reference as if each document were incorporated separately by reference. Furthermore, it should be understood that after reading the foregoing teachings of the present invention, various changes or modifications may be made to the present invention by those skilled in the art and that these equivalents also fall in the scope of the claims appended to this application.

1. An amino acid sequence that can destroy cells, wherein the destruction of the cells refers to the effect of triggering the collapse of the cell membrane system to destroy the cells, and can further comprise the effect of tissue damage caused by cell destruction,
and wherein the amino acid sequence comprises the amino acid sequence shown in SEQ ID NO: 1 and/or SEQ ID NO: 29.

2. (canceled)

3. The amino acid sequence of claim 1, wherein the amino acid sequence is selected from the following group:
(1) an Mdpcd1-303 protein fragment comprising the amino acid sequence shown in SEQ ID NO: 2;
(2) a derived protein or homologous protein of the Mdpcd1-303 protein fragment derived from SEQ ID NO: 2 with the same activity as the amino acid residue sequence of SEQ ID NO: 2, which is formed by substituting, deleting or adding one or more amino acid residues in the amino acid residue sequence of SEQ ID NO: 2.

4. The amino acid sequence of claim 3, wherein the derived protein is selected from the following group: an Mdpcd1-18 protein fragment, an Mdpcd1-297 protein fragment, an Mdpcd1-307 protein fragment, and combinations thereof,
the homologous protein comprises a Ptpcd1-296 protein fragment,
and wherein the Mdpcd1-18 protein fragment has the amino acid sequence shown in SEQ ID NO: 3;
the Mdpcd1-297 protein fragment has the amino acid sequence shown in SEQ ID NO: 4;
the Ptpcd1-296 protein fragment has the amino acid sequence shown in SEQ ID NO: 5;
and the Mdpcd1-307 protein fragment has the amino acid sequence shown in SEQ ID NO: 30.

5. A nucleotide sequence that can destroy cells, wherein the nucleotide sequence is used for encoding and obtaining an amino acid sequence that can destroy cells, and wherein the amino acid sequence is the amino acid sequence of claim 1.

6. The nucleotide sequence of claim 5, wherein the nucleotide sequence is used for encoding the Mdpcd1-303 protein fragment, the derived protein of the protein fragment, the homologous protein of the protein fragment, and combinations thereof.

7. The nucleotide sequence of claim 6, wherein the nucleotide sequence is used for encoding the Mdpcd1-303 protein fragment, and the nucleotide sequence has the nucleotide sequence shown in SEQ ID NO: 6.

8. The nucleotide sequence of claim 6, wherein the derived protein of the Mdpcd1-303 protein fragment comprises an Mdpcd1-18 protein fragment, an Mdpcd1-297 protein fragment, and an Mdpcd1-307 protein fragment, and the homologous protein comprises a Ptpcd1-296 protein fragment,
and wherein the nucleotide sequence for encoding the Mdpcd1-18 protein fragment is the nucleotide sequence shown in SEQ ID NO: 7,
the nucleotide sequence for encoding the Mdpcd1-297 protein fragment is the nucleotide sequence shown in SEQ ID NO: 8,
the nucleotide sequence for encoding the Ptpcd1-296 protein fragment is the nucleotide sequence shown in SEQ ID NO: 9,
and the nucleotide sequence for encoding the Mdpcd1-307 protein fragment is the nucleotide sequence shown in SEQ ID NO: 31.

9. A vector comprising the nucleotide sequence of claim 5.

10. A method for destroying cells comprising a step of using an amino acid sequence, a nucleotide sequence encoding the amino acid sequence, or a vector comprising the nucleotide sequence, wherein the amino acid sequence is the amino acid sequence of claim 1.

11. A method for tumor treatment comprising a step of using an amino acid sequence, a nucleotide sequence encoding the amino acid sequence, or a vector comprising the nucleotide sequence, wherein the amino acid sequence is the amino acid sequence of claim 1.

12-13. (canceled)

14. A composition for destroying cells, which comprises: an amino acid sequence, a nucleotide sequence encoding the amino acid sequence, or a vector comprising the nucleotide sequence, wherein the amino acid sequence is the amino acid sequence of claim 1.

15. A pharmaceutical composition for use in tumor treatment, which comprises:
an amino acid sequence, a nucleotide sequence encoding the amino acid sequence, or a vector comprising the nucleotide sequence, wherein the amino acid sequence is the amino acid sequence of claim 1.

16. The method of claim 10, wherein the method comprises the following step: (a) contacting the cells to be destroyed with the amino acid sequence, so as to collapse the cell membrane system of the cells, thereby destroying the cells.

17. The method of claim 10, wherein the cells are mammalian cells.

18. The method of claim 10, wherein the cells are tumor cells.

19. The method of claim 16, wherein in step (a), a nucleic acid or a vector expressing the amino acid sequence introduced into the cells, thereby expressing or overexpressing the amino acid sequence in the cells.

20. The method of claim 16, wherein the method further comprises the following step: (b) detecting the integrity of the cell membrane and/or the survival of the cells in step (a), thereby qualitatively or quantitatively determining the destruction of the cells.

21. The method of claim 10, wherein the method is an in vitro method.
